(19) United States
(12) Patent Application Publication
(10) Pub. No.: US 2024/0425486 A1
(43) Pub. Date: Dec. 26, 2024

(54) FUSED BICYCLIC SUBSTITUTED N-(1H-INDOL-7-YL)BENZENESULFONAMIDES AND USES THEREOF

(71) Applicant: Triana Biomedicines, Inc., Lexington, MA (US)

(72) Inventors: William J. Greenlee, Teaneck, NJ (US); Nicholas Calandra, Weston, MA (US); Soumya Ray, Quincy, MA (US)

(21) Appl. No.: 18/699,461

(22) PCT Filed: Oct. 7, 2022
(86) PCT No.: PCT/US2022/046088

§ 371 Date: Apr. 8, 2024

Related U.S. Application Data
(60) Provisional application No. 63/254,007, filed on Oct. 8, 2021.

(51) Int. Cl. C07D 403/12; A61K 31/404; A61K 31/4184; A61K 31/423; A61K 31/428; A61K 31/4365; A61K 31/4709; A61K 31/4725; A61K 31/517; A61K 31/536; A61K 31/538; A61K 31/5383; A61K 31/5415; A61K 31/55; A61K 31/553; A61K 31/554; C07D 209/30; C07D 409/12; C07D 413/12; C07D 417/12; C07D 495/04; C07D 498/04

(52) U.S. Cl. CPC C07D 403/12; A61K 31/404; A61K 31/4184; A61K 31/423; A61K 31/428; A61K 31/4365; A61K 31/4709; A61K 31/4725; A61K 31/517; A61K 31/536; A61K 31/538; A61K 31/5383; A61K 31/5415; A61K 31/55; A61K 31/553; A61K 31/554; C07D 209/30; C07D 409/12; C07D 413/12; C07D 417/12; C07D 495/04; C07D 498/04

(57) ABSTRACT

The present application discloses novel compounds, pharmaceutical compositions containing these compounds and methods of inducing degradation of a protein, comprising contacting the protein with an effective amount of a compound of the disclosure. Methods of treating disease and disorders that results from abnormal activity of a target protein in a subject, are also disclosed.

### RELATED APPLICATIONS

This application claims the benefit of priority to U.S. Provisional Application No. 63/254,007, filed Oct. 8, 2021, the entire contents of which are incorporated herein by reference.

### SUMMARY OF THE INVENTION

Various embodiments provide novel compounds, pharmaceutical compositions comprising such compounds, and methods of inducing degradation of a protein.

Some embodiments of the present disclosure describe a compound Formula I

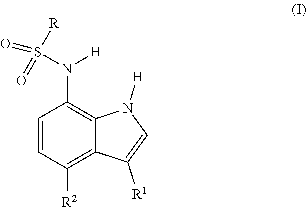

or a pharmaceutically acceptable salt thereof, wherein:
   R is an optionally substituted -5,6; -6,5; -6,6; or -6,7 bicyclic ring system;
   R¹ is selected from the group consisting of chloro, bromo, fluoro, and iodo; and
   R² is selected from the group consisting of H, chloro, fluoro, and methyl.

Some embodiments of the present disclosure are directed to a compound of Formula I, wherein
R is

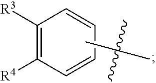

and
R³ and R⁴, together with the carbon atoms to which they are attached, form a 5 to 7-membered optionally substituted carbocyclic, heterocyclic, heteroaryl, or aryl ring.

Some embodiments of the present disclosure are directed to a compound of Formula I, or a pharmaceutically acceptable salt thereof, wherein:
R is

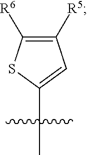

and
R⁵ and R⁶, together with the carbon atoms to which they are attached, form an optionally substituted 6-membered heterocyclylic or aryl ring.

Some embodiments of the present disclosure are directed to a compound selected from the group consisting of

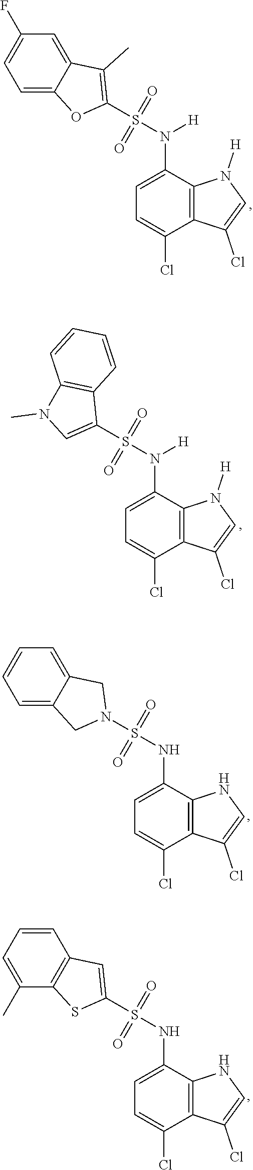 [SMILES: Cc1c(S(=O)(=O)Nc2ccc(Cl)c3c(Cl)c[nH]c23)oc2ccc(F)cc12 ; Cn1cc(S(=O)(=O)Nc2ccc(Cl)c3c(Cl)c[nH]c23)c2ccccc21 ; O=S(=O)(Nc1ccc(Cl)c2c(Cl)c[nH]c12)N1Cc2ccccc2C1 ; Cc1cccc2cc(S(=O)(=O)Nc3ccc(Cl)c4c(Cl)c[nH]c34)sc12]

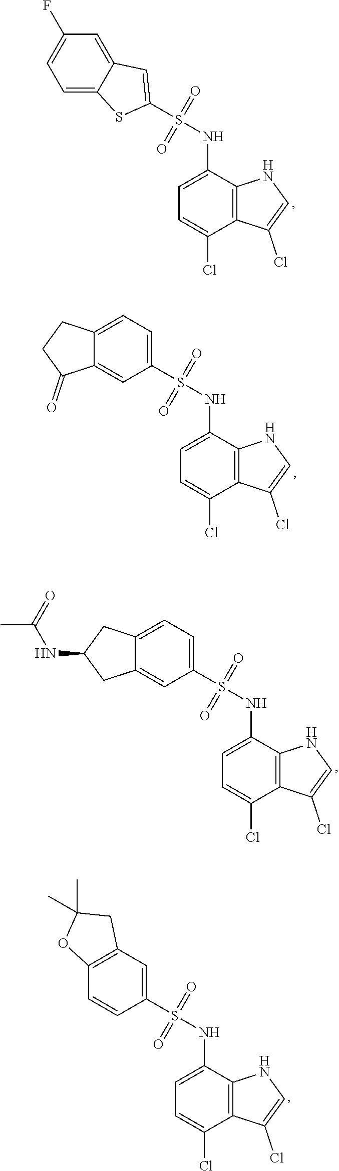 [SMILES: O=S(=O)(Nc1ccc(Cl)c2c(Cl)c[nH]c12)c1cc2cc(F)ccc2s1 ; O=C1CCc2ccc(S(=O)(=O)Nc3ccc(Cl)c4c(Cl)c[nH]c34)cc21 ; CC(=O)N[C@H]1Cc2ccc(S(=O)(=O)Nc3ccc(Cl)c4c(Cl)c[nH]c34)cc2C1 ; CC1(C)Cc2cc(S(=O)(=O)Nc3ccc(Cl)c4c(Cl)c[nH]c34)ccc2O1]

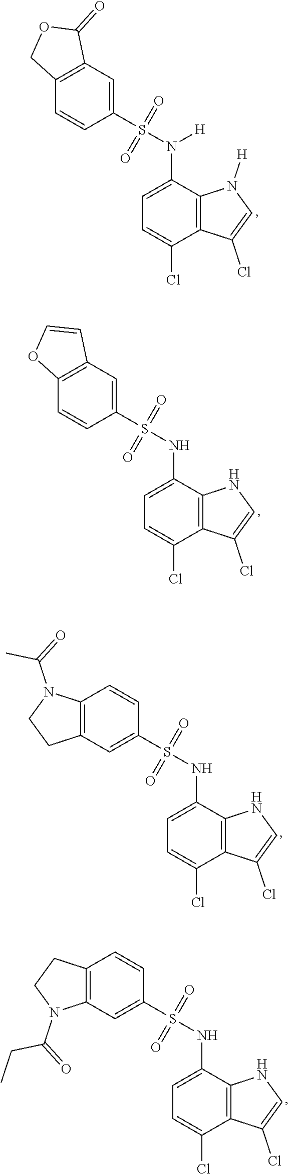 [SMILES: O=C1OCc2ccc(S(=O)(=O)Nc3ccc(Cl)c4c(Cl)c[nH]c34)cc21 ; O=S(=O)(Nc1ccc(Cl)c2c(Cl)c[nH]c12)c1ccc2occc2c1 ; CC(=O)N1CCc2cc(S(=O)(=O)Nc3ccc(Cl)c4c(Cl)c[nH]c34)ccc21 ; CCC(=O)N1CCc2ccc(S(=O)(=O)Nc3ccc(Cl)c4c(Cl)c[nH]c34)cc21]

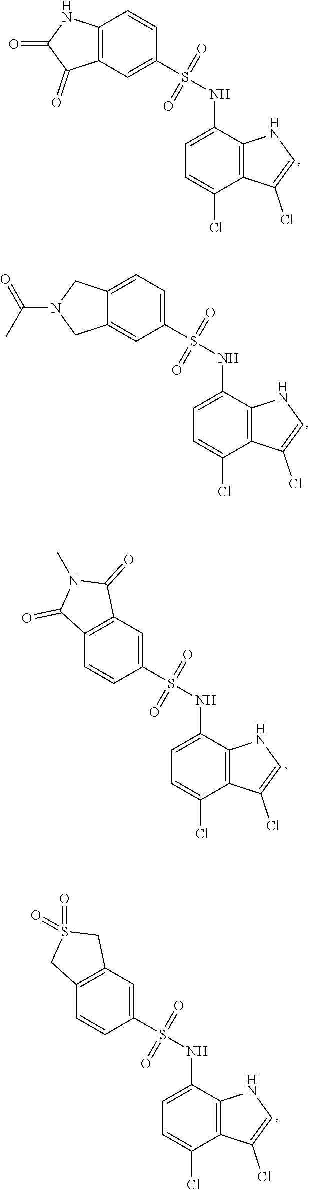 [SMILES: O=C1Nc2ccc(S(=O)(=O)Nc3ccc(Cl)c4c(Cl)c[nH]c34)cc2C1=O ; CC(=O)N1Cc2ccc(S(=O)(=O)Nc3ccc(Cl)c4c(Cl)c[nH]c34)cc2C1 ; CN1C(=O)c2ccc(S(=O)(=O)Nc3ccc(Cl)c4c(Cl)c[nH]c34)cc2C1=O ; O=S1(=O)Cc2ccc(S(=O)(=O)Nc3ccc(Cl)c4c(Cl)c[nH]c34)cc2C1]

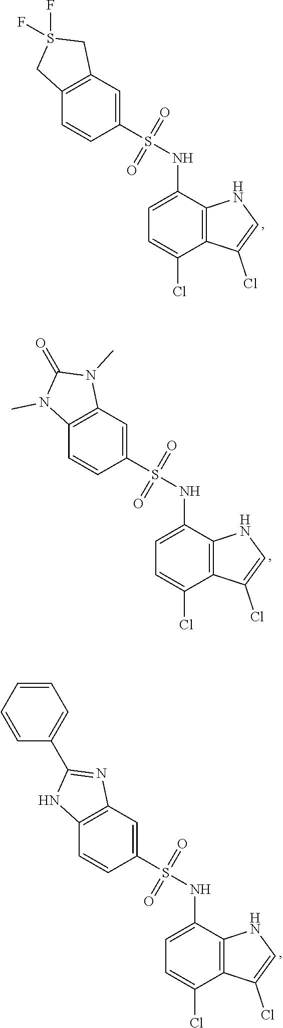 [SMILES: O=S(=O)(Nc1ccc(Cl)c2c(Cl)c[nH]c12)c1ccc2c(c1)CS(F)(F)C2 ; Cn1c(=O)n(C)c2cc(S(=O)(=O)Nc3ccc(Cl)c4c(Cl)c[nH]c34)ccc21 ; O=S(=O)(Nc1ccc(Cl)c2c(Cl)c[nH]c12)c1ccc2[nH]c(-c3ccccc3)nc2c1]

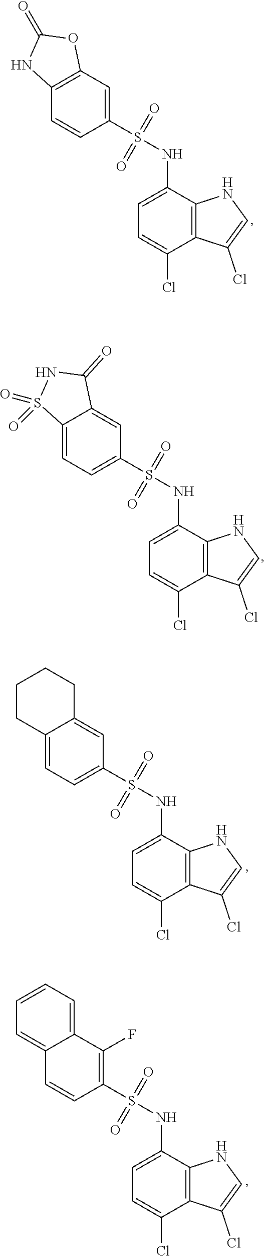 [SMILES: O=c1[nH]c2ccc(S(=O)(=O)Nc3ccc(Cl)c4c(Cl)c[nH]c34)cc2o1 ; O=C1NS(=O)(=O)c2ccc(S(=O)(=O)Nc3ccc(Cl)c4c(Cl)c[nH]c34)cc21 ; O=S(=O)(Nc1ccc(Cl)c2c(Cl)c[nH]c12)c1ccc2c(c1)CCCC2 ; O=S(=O)(Nc1ccc(Cl)c2c(Cl)c[nH]c12)c1ccc2ccccc2c1F]

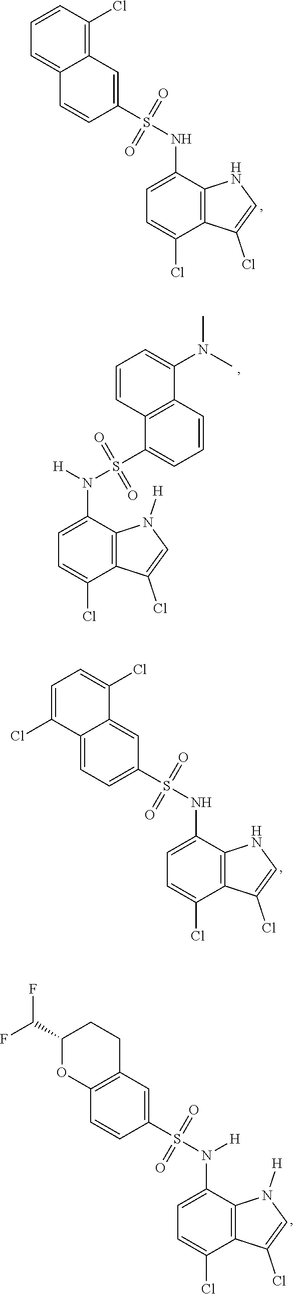 [SMILES: O=S(=O)(Nc1ccc(Cl)c2c(Cl)c[nH]c12)c1ccc2cccc(Cl)c2c1 ; CN(C)c1cccc2c(S(=O)(=O)Nc3ccc(Cl)c4c(Cl)c[nH]c34)cccc12 ; O=S(=O)(Nc1ccc(Cl)c2c(Cl)c[nH]c12)c1ccc2c(Cl)ccc(Cl)c2c1 ; O=S(=O)(Nc1ccc(Cl)c2c(Cl)c[nH]c12)c1ccc2c(c1)CC[C@@H](C(F)F)O2]

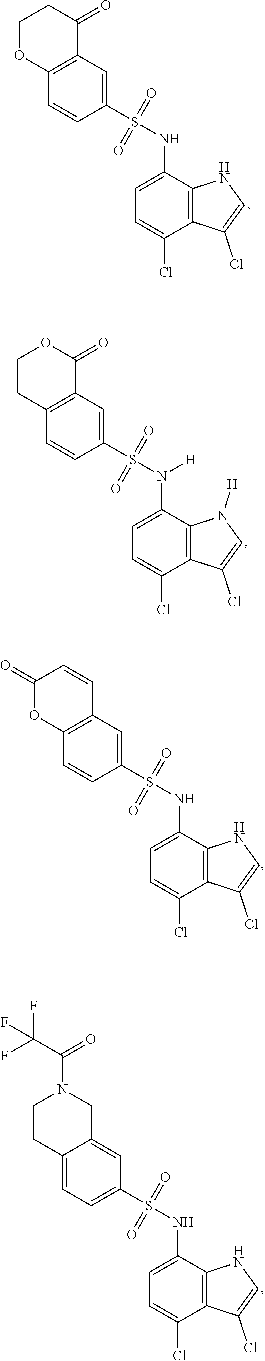 [SMILES: O=C1CCOc2ccc(S(=O)(=O)Nc3ccc(Cl)c4c(Cl)c[nH]c34)cc21 ; O=C1OCCc2ccc(S(=O)(=O)Nc3ccc(Cl)c4c(Cl)c[nH]c34)cc21 ; O=c1ccc2cc(S(=O)(=O)Nc3ccc(Cl)c4c(Cl)c[nH]c34)ccc2o1 ; O=C(N1CCc2ccc(S(=O)(=O)Nc3ccc(Cl)c4c(Cl)c[nH]c34)cc2C1)C(F)(F)F]

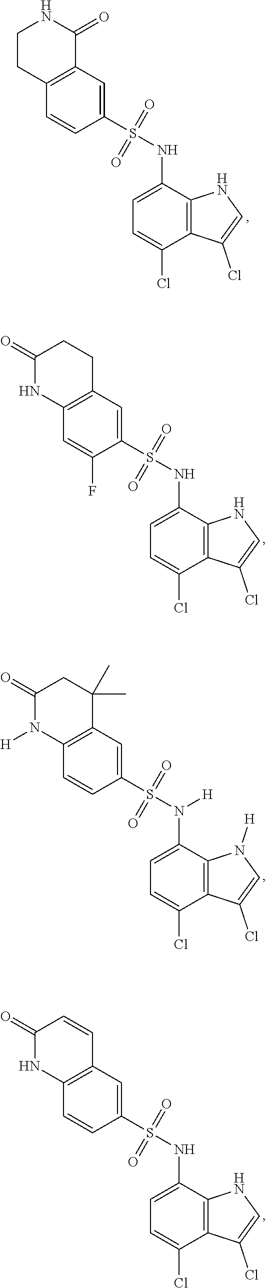 [SMILES: O=C1NCCc2ccc(S(=O)(=O)Nc3ccc(Cl)c4c(Cl)c[nH]c34)cc21 ; O=C1CCc2cc(S(=O)(=O)Nc3ccc(Cl)c4c(Cl)c[nH]c34)c(F)cc2N1 ; CC1(C)CC(=O)Nc2ccc(S(=O)(=O)Nc3ccc(Cl)c4c(Cl)c[nH]c34)cc21 ; O=c1ccc2cc(S(=O)(=O)Nc3ccc(Cl)c4c(Cl)c[nH]c34)ccc2[nH]1]

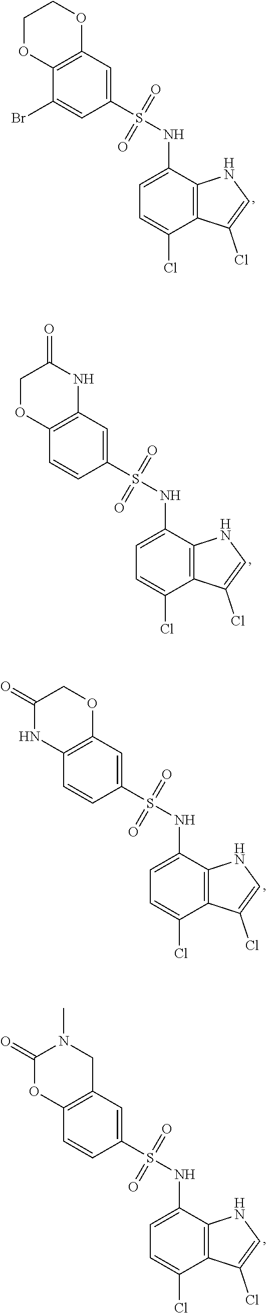 [SMILES: O=S(=O)(Nc1ccc(Cl)c2c(Cl)c[nH]c12)c1cc(Br)c2c(c1)OCCO2 ; O=C1COc2ccc(S(=O)(=O)Nc3ccc(Cl)c4c(Cl)c[nH]c34)cc2N1 ; O=C1COc2cc(S(=O)(=O)Nc3ccc(Cl)c4c(Cl)c[nH]c34)ccc2N1 ; CN1Cc2cc(S(=O)(=O)Nc3ccc(Cl)c4c(Cl)c[nH]c34)ccc2OC1=O]

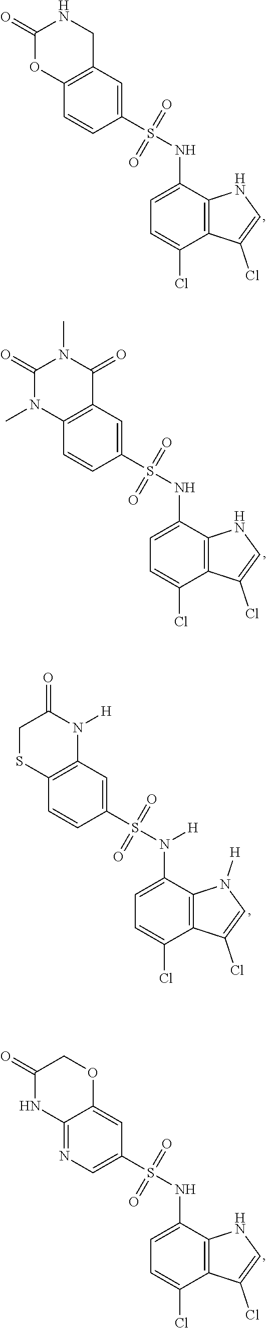 [SMILES: O=C1NCc2cc(S(=O)(=O)Nc3ccc(Cl)c4c(Cl)c[nH]c34)ccc2O1 ; Cn1c(=O)c2cc(S(=O)(=O)Nc3ccc(Cl)c4c(Cl)c[nH]c34)ccc2n(C)c1=O ; O=C1CSc2ccc(S(=O)(=O)Nc3ccc(Cl)c4c(Cl)c[nH]c34)cc2N1 ; O=C1COc2cc(S(=O)(=O)Nc3ccc(Cl)c4c(Cl)c[nH]c34)cnc2N1]

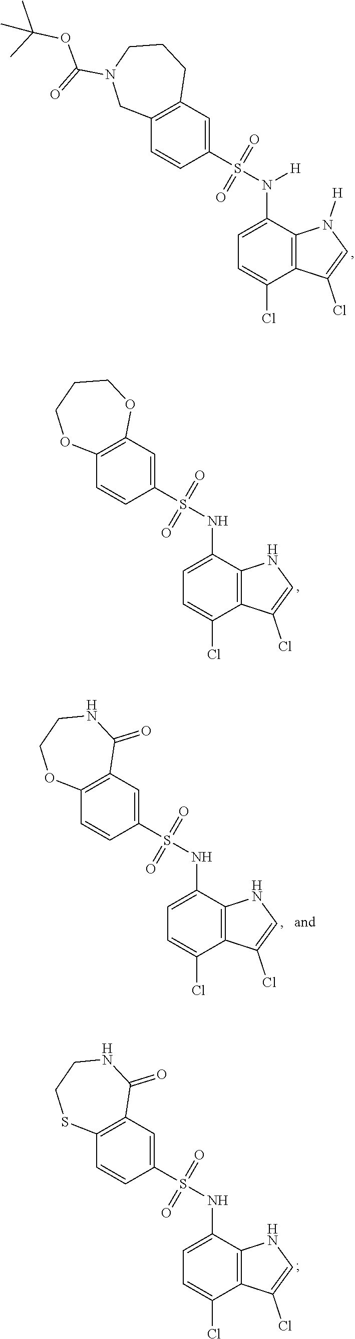 [SMILES: CC(C)(C)OC(=O)N1CCCc2cc(S(=O)(=O)Nc3ccc(Cl)c4c(Cl)c[nH]c34)ccc2C1 ; O=S(=O)(Nc1ccc(Cl)c2c(Cl)c[nH]c12)c1ccc2c(c1)OCCCO2 ; O=C1NCCOc2ccc(S(=O)(=O)Nc3ccc(Cl)c4c(Cl)c[nH]c34)cc21 ; O=C1NCCSc2ccc(S(=O)(=O)Nc3ccc(Cl)c4c(Cl)c[nH]c34)cc21]

or a pharmaceutically acceptable salt thereof.

Some embodiments of the present disclosure are directed to a pharmaceutical composition comprising a therapeutically effective amount of a compound of described herein and a pharmaceutically acceptable carrier or diluent.

Some embodiments of the present disclosure are directed to a method of inducing degradation of a protein, comprising contacting the protein with an effective amount of a compound or composition according to embodiments described herein.

Some embodiments of the present disclosure are directed to a method of inducing degradation of a protein, in a patient in need thereof, comprising administering to the patient, a therapeutically effective amount of a compound according to embodiments herein.

Some embodiments of the present disclosure are directed to a method of treating a disease or disorder that results from abnormal activity of a target protein in a subject, comprising administering to the subject, a therapeutically effective amount of a compound according to embodiments herein, wherein the compound induces degradation of the protein thereby treating the disease or disorder.

Some embodiments of the present disclosure are directed to a method of treating a disease or disorder that results from abnormal expression of a gene that encodes a target protein in a subject, comprising administering to the subject, a therapeutically effective amount of a compound according to embodiments herein, wherein the compound induces degradation of the protein thereby treating the disease or disorder.

### DETAILED DESCRIPTION

Molecular glues are small molecules that bring about targeted protein degradation by promoting the association of a target protein with a ubiquitin E3 ligase. This results in ubiquitination of the protein, followed by its degradation by the proteasome. During protein degradation, the molecular glue dissociates, freeing it up to form a new target protein-E3 ligase complex. Examples of molecular glues include the IMiDs (immune modulatory drugs; e.g., thalidomide), which promote a novel interaction between a substrate (e.g., IKZF1/3) and cereblon, a substrate receptor (also known as DCAF) for Cullin-RING ubiquitin ligase 4 (CRL4). More recently, the small molecule indisulam was reported to be a molecular glue that enhances the binding of DCAF15, another CRL4 substrate receptor, to a novel substrate, the pre-mRNA splicing factor RBM39, promoting its degradation.

Molecular glues offer advantages as pharmaceuticals for treating a variety of diseases and disorders. By targeting specific proteins in a cell and eliminating or limiting their function, they can provide highly specific drug therapy options with fewer side effects, such as off target effects and/or toxicity. Furthermore, more than three quarters of human proteins have remained beyond the reach of therapeutic development, despite enormous efforts to advance traditional pharmacology approaches. Compounds that bring about targeted protein degradation offer one approach that can overcome this limitation. Thus, there is a need for new compounds that function as molecular glues and induce protein degradation for the treatment of diseases and disorders.

### Definitions

Unless defined otherwise, all technical and scientific terms used herein have the same meanings as commonly understood by one of ordinary skill in the art.

The term “−5,6 bicyclic ring system” or as defined herein, is a bicyclic ring system having a five-membered ring and six-membered ring joined together, whereby the joined ring system has a point of attachment “-” on the five-membered ring. Each ring in the bicyclic ring can be carbocyclic or heterocyclic and can be aromatic or aliphatic or a combination thereof.

The term “-6,5 bicyclic ring system” as defined herein, is a bicyclic ring system having a six-membered ring and a five-membered ring, whereby the ring system has a point of attachment “-” on the six-membered ring. Each ring in the bicyclic ring can be carbocyclic or heterocyclic and can be aromatic or aliphatic or a combination thereof.

The term “-6,6 bicyclic ring system” as defined herein, is a bicyclic ring system having two six-membered rings whereby the ring system has a point of attachment “-” on one of the six-membered rings. Each ring in the bicyclic ring can be carbocyclic or heterocyclic and can be aromatic or aliphatic or a combination thereof.

The term “-6,7 bicyclic ring system” as defined herein, is a bicyclic ring system having a six-membered ring and a seven-membered ring, whereby the bicyclic ring system has a point of attachment “-” on the six-membered ring. Each ring in the bicyclic ring can be carbocyclic or heterocyclic and can be aromatic or aliphatic or a combination thereof.

The articles “a” and “an” as used herein mean “one or more” or “at least one,” unless otherwise indicated. That is, reference to any element of the present invention by the indefinite article “a” or “an” does not exclude the possibility that more than one of the element is present.

As used herein, the term “about” means plus or minus 10% of the numerical value of the number with which it is being used. Therefore, about 50% means in the range of 45%-55%.

“Administering,” or “administration” and the like, when used in conjunction with the compounds of the disclosure refers to providing the compounds or pharmaceutical compositions according to any of the embodiments described herein, to a subject in need of treatment. Preferably the subject is a mammal, more preferably a human. The present invention comprises administering the compound or pharmaceutical composition of the invention alone or in conjunction with another therapeutic agent. When a compound or pharmaceutical composition of the invention is administered in conjunction with another therapeutic agent, the compound or pharmaceutical composition of the invention and the other therapeutic agent can be administered at the same time or different times, and by the same routes of administration or by different routes of administration.

As used herein, the term “alkyl” is intended to include both branched and straight-chain saturated aliphatic hydrocarbon groups having the specified number of carbon atoms. For example, “C₁-C₁₇alkyl” or “C₁₋₁₇ alkyl” (or alkylene), is intended to include C₁, C₂, C₃, C₄, C₅, C₆, C₇, C₈, C₉, C₁₀, C₁₁, C₁₂, C₁₃, C₁₄, C₁₅, C₁₆, and C₁₇ alkyl groups. Additionally, for example, “C₁-C₆ alkyl” or “C₁₋₆ alkyl” denotes alkyl having 1 to 6 carbon atoms.

The term “haloalkyl” includes mono, poly, and perhaloalkyl groups where the halogens are independently selected from fluorine, chlorine, bromine, and iodine.

The term “alkoxy” refers to an —O-alkyl group. “C₁-C₆ alkoxy” or “C₁₋₆ alkoxy” (or alkyloxy), is intended to include C₁, C₂, C₃, C₄, C₅, and C₆, alkoxy groups. Examples of alkoxy groups include, but are not limited to, methoxy, ethoxy, propoxy (e.g., n-propoxy and isopropoxy), and t-butoxy.

The term “haloalkoxy” includes mono, poly, and perhaloalkoxy groups where the halogens are independently selected from fluorine, chlorine, bromine, and iodine.

The term “animal” as used herein includes, but is not limited to, humans and non-human vertebrates such as wild, domestic and farm animals.

“Aryl” groups refer to monocyclic or polycyclic aromatic hydrocarbons, including, for example, phenyl, and naphthyl. “C₆-C₁₀ aryl” or “C₆₋₁₀ aryl” refers to phenyl and naphthyl.

Unless otherwise indicated, the term “compound” refers to any compound described herein. In certain aspects, where specified, one or more hydrogen atoms on a disclosed compound may be replaced with deuterium. Such deuterated compounds may have one or more improved pharmacokinetic or pharmacodynamic properties (e.g., longer half-life) compared to the equivalent “un-deuterated” compound.

The term “cycloalkyl” or “cycloalkyl ring” is defined as a saturated or partially unsaturated carbocyclic ring in a single or fused carbocyclic ring system (i.e., a ring system composed of all carbon atoms) having from three to twelve ring members. In a preferred embodiment, a cycloalkyl is a ring system having three to seven ring members.

The term “halo” or “halogen” refers to fluoro, chloro, bromo, and iodo.

As used herein, the term “heteroaryl” is intended to mean stable monocyclic and polycyclic aromatic hydrocarbons that include at least one heteroatom ring member, such as sulfur, oxygen, or nitrogen. Heteroaryl groups include, without limitation, pyridinyl, pyrimidinyl, pyrazinyl, pyridazinyl, triazinyl, furanyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-oxadiazolyl, 1,2,4-thiadiazolyl, isothiazolyl, purinyl, carbazolyl, benzimidazolyl, indolinyl, benzodioxolanyl, and benzodioxane.

The term “heterocyclyl” means, unless otherwise specified, a 4- to 12-membered saturated or partially unsaturated heterocyclic ring containing 1 to 4 heteroatoms independently selected from N, O, and S. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, 7-azaspiro[3.5]nonanyl, 6-azaspiro[3.4]octanyl and the like.

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “optionally substituted” means that that one or more hydrogens of the designated moiety may be replaced with a suitable substituent. Unless otherwise indicated, an “optionally substituted” group may have a suitable substituent at each substitutable position of the group as valency permits. Optional substituents include, but are not limited to, one or more groups selected from cyano (—CN), halo, imino (═NH), nitro (—NO₂), oxo (═O), —C(O)Rⁱ, —C(O)ORⁱ, —C(O)NRⁱⁱRⁱⁱⁱ, —C(O)SRⁱ, —C(NRⁱ)NRⁱⁱRⁱⁱⁱ, —C(S)Rⁱ, —C(S)ORⁱ, —C(S)NRⁱⁱRⁱⁱⁱ, —ORⁱ, —OC(O)Rⁱ, —OC(O)ORⁱ, —OC(O)NRⁱⁱRⁱⁱⁱ, —OC(O)SRⁱ, —OC(NRⁱ)NRⁱⁱRⁱⁱⁱ, —OC(S)Rⁱ, —OC(S)ORⁱ, —OC(S)NRⁱⁱRⁱⁱⁱ, —OP(O)(ORⁱⁱ)ORⁱⁱⁱ, —OS(O)Rⁱ, —OS(O)₂Rⁱ, —OS(O)NRⁱⁱRⁱⁱⁱ, —OS(O)₂NRⁱⁱRⁱⁱⁱ, —NRⁱⁱRⁱⁱⁱ, —NRⁱC(O)Rᵢᵥ, NRⁱC(O)OR^{iv}, NRⁱC(O)NRⁱⁱRⁱⁱⁱ, —NR^{a}C(O)SR^{iv}, —NRⁱC(NR^{iv})NRⁱⁱRⁱⁱⁱ, —NRⁱC(S)Rᵢᵥ, —NRⁱC(S)OR^{iv}, —NRⁱC(S)NRⁱⁱRⁱⁱⁱ, —NRⁱS(O)R^{iv}, —NRⁱS(O)₂R^{iv}, —NRⁱS(O)NRⁱⁱRⁱⁱⁱ, —NRⁱS(O)₂NRⁱⁱR^{iv}, —SRⁱ, —S(O)Rⁱ, —S(O)₂Rⁱ, —S(O)NRⁱⁱR^{iv}, —S(O)₂NRⁱⁱR^{iv}, alkyl, heteroalkyl, alkenyl, alkynyl, cycloalkyl, aryl, heteroaryl, and heterocyclyl, wherein said alkyl, heteroalkyl, alkenyl, alkynyl, cycloalkyl, aryl, heteroaryl, and heterocyclyl are each further optionally substituted with one or more, in one embodiment, one, two, three, or four, substituents Q^{a}, wherein each Rⁱ, Rⁱⁱ, Rⁱⁱⁱ, and R^{iv} is independently alkyl, heteroalkyl, alkenyl, alkynyl, cycloalkyl, aryl, heteroaryl, or heterocyclyl, each of which is optionally substituted with one or more, in one embodiment, one, two, three, or four, substituents Q^{a} or Rⁱⁱ and Rⁱⁱⁱ together with the N atom to which they are attached form heterocyclyl optionally substituted with one or more, in one embodiment, one, two, three, or four, substituents Q^{a}, wherein each Q^{a} is independently selected from cyano, halo, imino, nitro, oxo, alkyl, heteroalkyl, alkenyl, alkynyl, cycloalkyl, C₆₋₁₄ aryl, heteroaryl, heterocyclyl, —C(O)R^{v}, —C(O)OR^{v}, —C(O)NR^{vi}R^{vii}, —C(O)SR^{v}, —C(NR^{v})NR^{vi}R^{vii}, —C(S)R^{v}, —C(S)OR^{v}, —C(S)NR^{vi}R^{vii}, —OR^{v}, —OC(O)R^{v}, —OC(O)OR^{v}, —OC(O)NR^{vi}R^{vii}, —OC(O)SR^{v}, —OC(NR^{v})NR^{vi}R^{vii}, —OC(S)R^{v}, —OC(S)OR^{v}, —OC(S)NR^{vi}R^{vii}, —OP(O)(OR^{v})OR^{vi}, —OS(O)R^{v}, —OS(O)₂R^{v}, —OS(O)NR^{vi}R^{vii}, —OS(O)₂NR^{v}R^{vii}, —NR^{vi}R^{vii}, —NR^{v}C(O)R^{vii}, —NR^{e}C(O)OR^{vi}, —NR^{v}C(O)NR^{vi}R^{vii}, —NR^{v}C(O)SR^{vi}, —NR^{v}C(NR^{viii})NR^{vi}R^{vii}, —NR^{v}C(S)R^{viii}, —NR^{v}C(S)OR^{vi}, —NR^{v}C(S)NR^{vi}R^{vii}, —NR^{v}S(O)R^{vii}, —NR^{v}S(O)₂R^{viii}, —NR^{v}S(O)NR^{vi}R^{vii}, —NR^{v}S(O)₂NR^{vi}R^{vii}, —SR^{v}, —S(O)R^{v}, —S(O)₂R^{v}, —S(O)NR^{vi}R^{vii}, and —S(O)₂NR^{vi}R^{vii}; wherein each R^{v}, R^{vi}, R^{vii}, and R^{viii} is independently alkyl, alkenyl, alkynyl, cycloalkyl, aryl, aralkyl, heteroaryl, or heterocyclyl; or (iii) R^{vi} and R^{vii} together with the N atom to which they are attached form heterocyclyl. In one aspect, the term “optionally substituted” means that that one or more hydrogens of the designated moiety may be replaced with halo, (C₁-C₄)alkyl, (C₁-C₄)haloalkyl, (C₁-C₄)alkoxy, (C₁-C₄)haloalkoxy, —C(O)R^{a}R^{b}, —C(O)OR^{a}R^{b}, —NR′C(O)R^{a}R^{b}, —NR′C(O)OR^{a}R^{b}, phenyl, oxo, —NR^{a}R^{b}, —C(O)NR^{a}R^{b}, and cyano, wherein R^{a}, R^{b}, and R′ are each independently selected from hydrogen, (C₁-C₄)alkyl, and (C₁-C₄)haloalkyl. In another aspect, the term “optionally substituted” means that that one or more hydrogens of the designated moiety may be replaced with halo, (C₁-C₄)alkyl, (C₁-C₄)haloalkyl, —C(O)R^{a}R^{b}, —C(O)OR^{a}R^{b}, —NR′C(O)R^{a}R^{b}, phenyl, oxo, and —NR^{a}R^{b}, wherein R^{a}, R^{b}, and R′ are each independently selected from hydrogen, (C₁-C₄)alkyl, and (C₁-C₄)haloalkyl. In yet another aspect, the term “optionally substituted” means that that one or more hydrogens of the designated moiety may be replaced with halo, (C₁-C₄)alkyl, —C(O)(C₁-C₄)alkyl, —C(O)(C₁-C₄)haloalkyl, oxo, —NHC(O)(C₁-C₄)alkyl, phenyl, N[(C₁-C₄)alkyl]₂, and —C(O)O(C₁-C₄)alkyl.

The phrase “molecular glue” as used herein is used to describe a compound that induces an interaction between a substrate receptor of an E3 ligase and a target protein, leading to degradation of the protein.

The phrase “pharmaceutically acceptable” refers to those compounds, materials, pharmaceutical compositions, and/or dosage forms that are, within the scope of sound medical judgment, generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal government or a state government or listed in the U.S. Pharmacopoeia or other generally recognized pharmacopoeia for use in animals, and more particularly in humans.

As used herein, the term “pharmaceutically acceptable carrier” is a substrate used to deliver the compounds of the invention. Carriers as used herein include excipients and diluents and may depend upon the mode of administration of the compounds as described herein. Carriers for example may be edible carriers, liquid carries, carriers that will protect the compounds against rapid elimination from the body, as well as liposomal suspensions. Carriers are well known in the art and include, for example, phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

The terms “subject,” “individual” or “patient” are used interchangeably and as used herein are intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients. The methods are particularly suitable for treating human patients having a condition, disease or disorder described herein.

As used herein, the term “therapeutic” means an agent utilized to treat, combat, ameliorate, or improve an unwanted condition or disease of a patient.

A “therapeutically effective amount” of a compound, pharmaceutically acceptable salt thereof or pharmaceutical composition according to any embodiment described herein, is an amount sufficient to produce a selected effect on at least one symptom or parameter of a specific disease or disorder. The therapeutic effect may be objective (i.e., measurable by some test or marker) or subjective (i.e., subject gives an indication of or feels an effect or physician observes a change). The effect contemplated herein, includes both medical therapeutic and/or prophylactic treatment, as appropriate. The specific dose of a compound administered according to this disclosure to obtain therapeutic and/or prophylactic effects is determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment. The therapeutically effective amount administered will be determined by the physician in the light of the foregoing relevant circumstances and the exercise of sound medical judgment. A therapeutically effective amount of a compound, according to any embodiment described herein, is typically an amount such that when it is administered in a physiologically tolerable excipient composition, it is sufficient to achieve an effective systemic concentration or local concentration in the tissue.

The terms “treat,” “treated,” or “treating” as used herein, refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment. In one aspect, treatment “treat,” “treated,” or “treating” as used herein, means therapeutic treatment.

### Compounds

Some embodiments of the present disclosure describe a compound Formula I

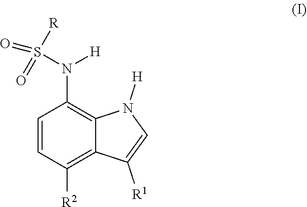

or a pharmaceutically acceptable salt thereof, wherein: R is an optionally substituted-5,6; -6,5; -6,6; or -6,7 bicyclic ring system and R¹ and R² are as defined herein.

In some embodiments R is a heteroaryl bicyclic ring or heterocyclic bicyclic ring.

In some embodiments R is an optionally substituted -5,6 bicyclic ring system. In some embodiments R is selected from the group consisting of optionally substituted:

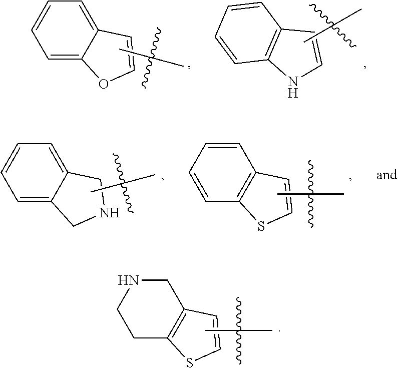

In some embodiments R is selected from the group consisting of

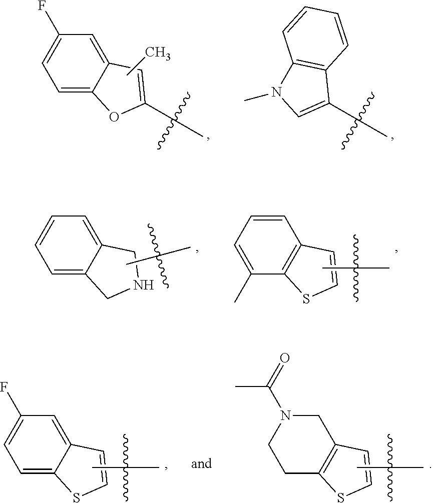

In some embodiments R is selected from the group consisting of:

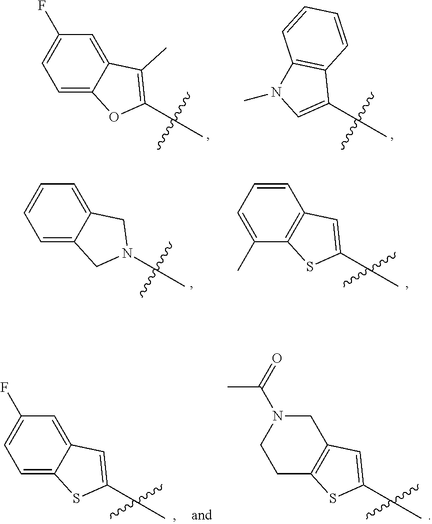 [SMILES: *c1oc2ccc(F)cc2c1C ; *c1cn(C)c2ccccc12 ; *N1Cc2ccccc2C1 ; *c1cc2cccc(C)c2s1 ; *c1cc2cc(F)ccc2s1 ; *c1cc2c(s1)CCN(C(C)=O)C2]

In some embodiments R is an optionally substituted -6,5 bicyclic ring system.

In some embodiments R is selected from the group consisting of optionally substituted:

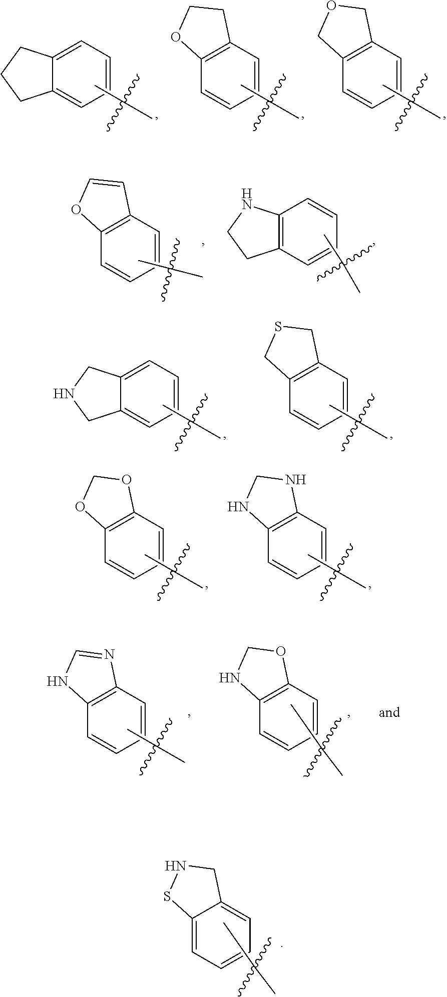

In some embodiments R is selected from the group consisting of

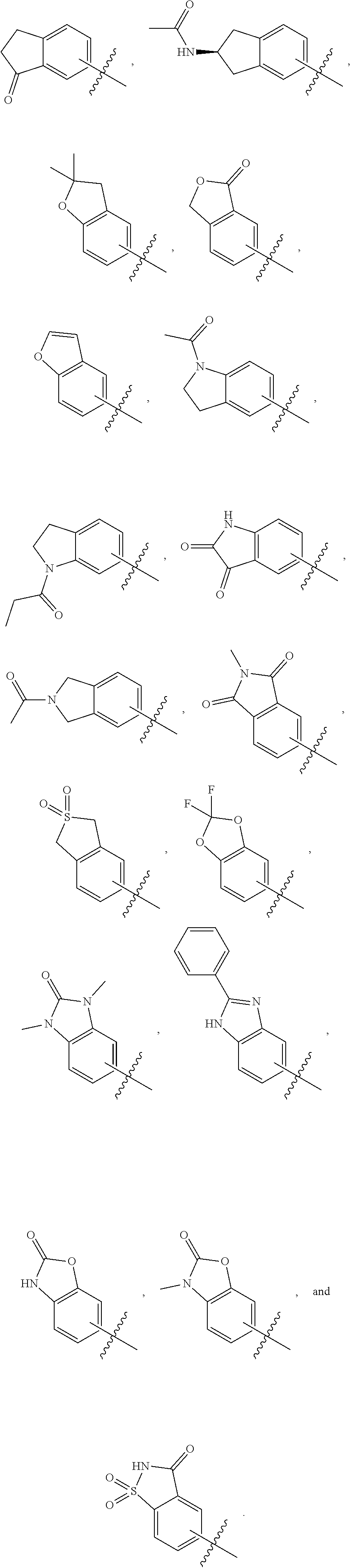

In some embodiments R is selected from the group consisting of:

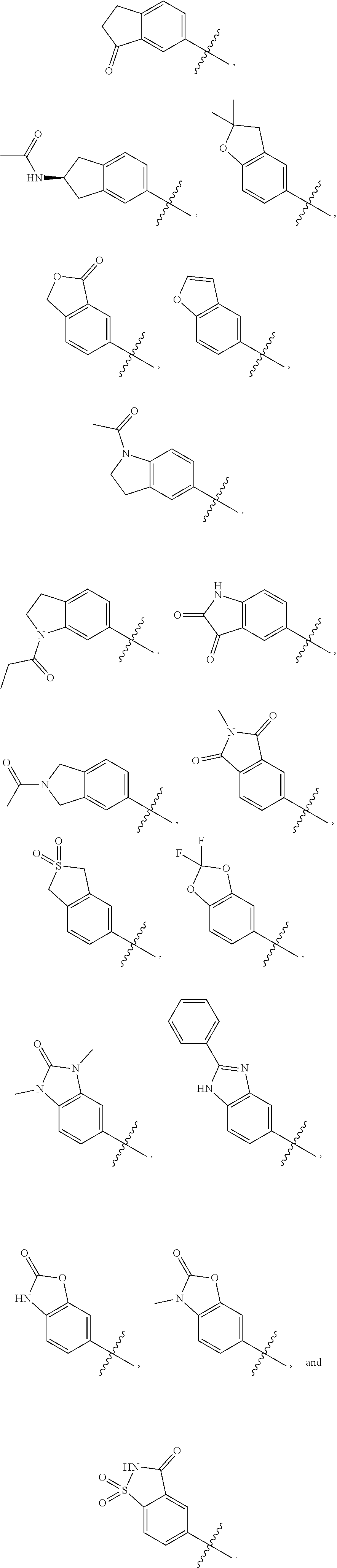

In some embodiments R is an optionally substituted -6,6 bicyclic ring system. In some embodiments R is selected from the group consisting of optionally substituted:

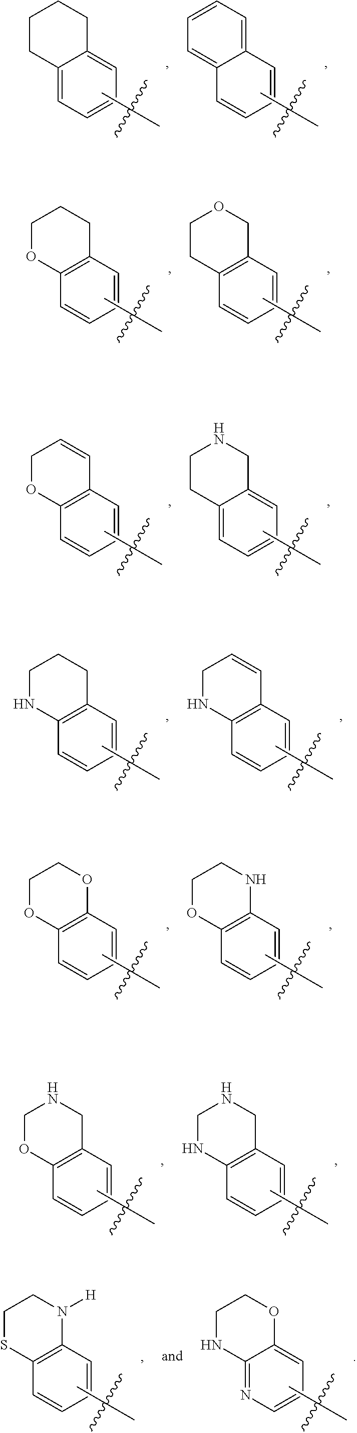

In some embodiments R is selected from the group consisting of

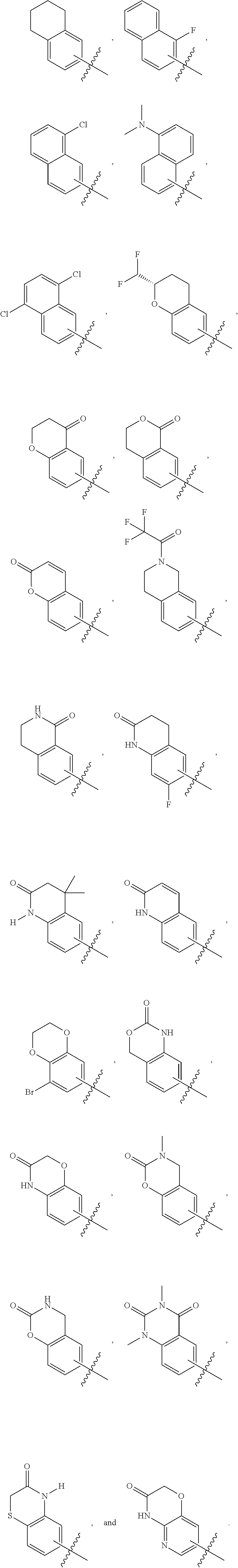

In some embodiments R is selected from the group consisting of

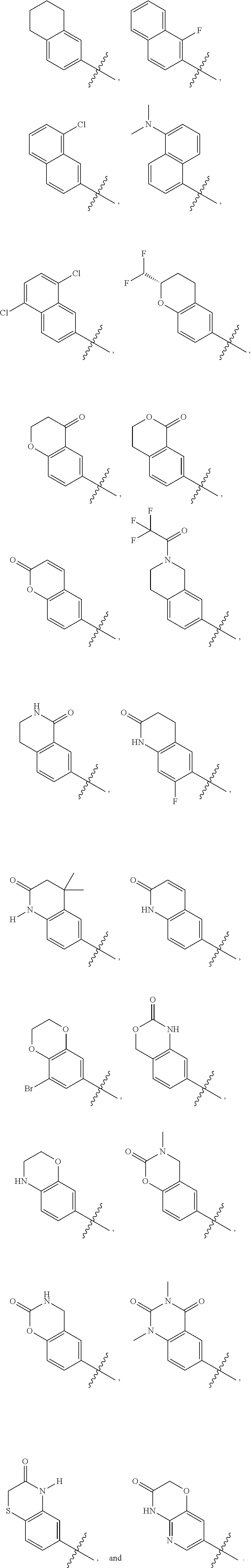

In some embodiments R is an optionally substituted -6,7 bicyclic ring system. In some embodiments R is selected from the group consisting of optionally substituted:

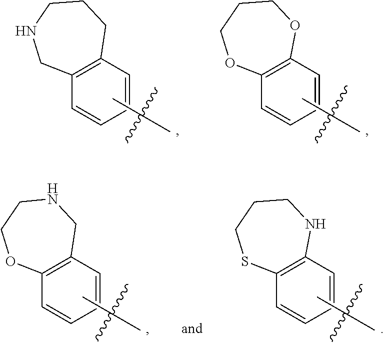

In some embodiments R is selected from the group consisting of:

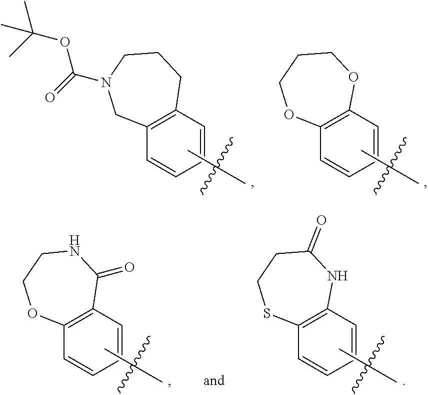

In some embodiments R is selected from the group consisting of:

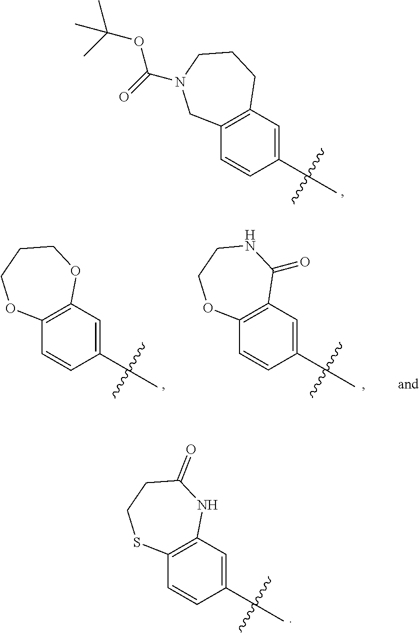 [SMILES: *c1ccc2c(c1)CCCN(C(=O)OC(C)(C)C)C2 ; *c1ccc2c(c1)OCCCO2 ; *c1ccc2c(c1)C(=O)NCCO2 ; *c1ccc2c(c1)NC(=O)CCS2]

In some embodiments R¹ of Formula I is selected from the group consisting of chloro, bromo, fluoro, and iodo. In some embodiments R¹ is chloro.

In some embodiments R² of Formula I is selected from the group consisting of H, chloro, fluoro, and methyl. In some embodiments R² is chloro.

In some embodiments each of R¹ and R² is chloro. In some embodiments each of R¹ and R² is chloro and R is selected from the group consisting of

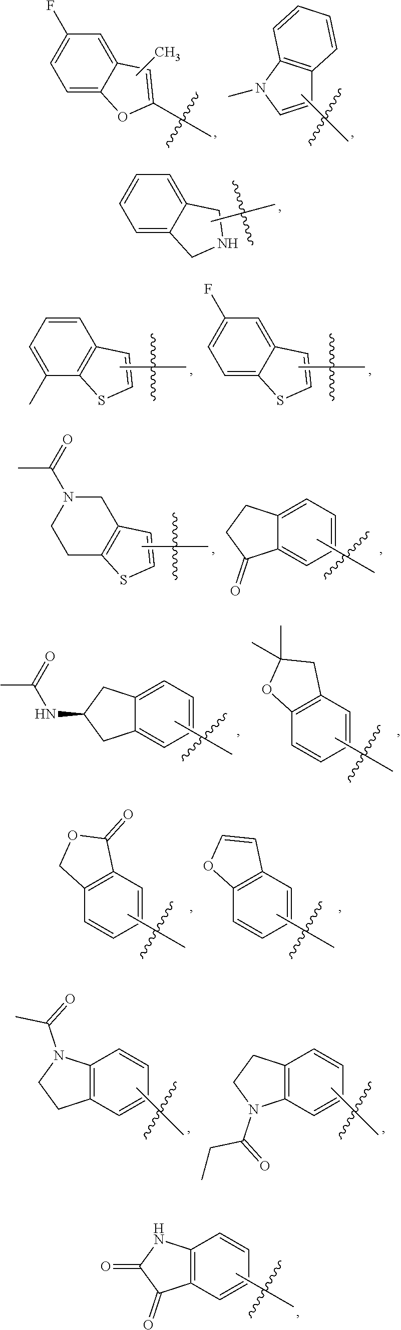

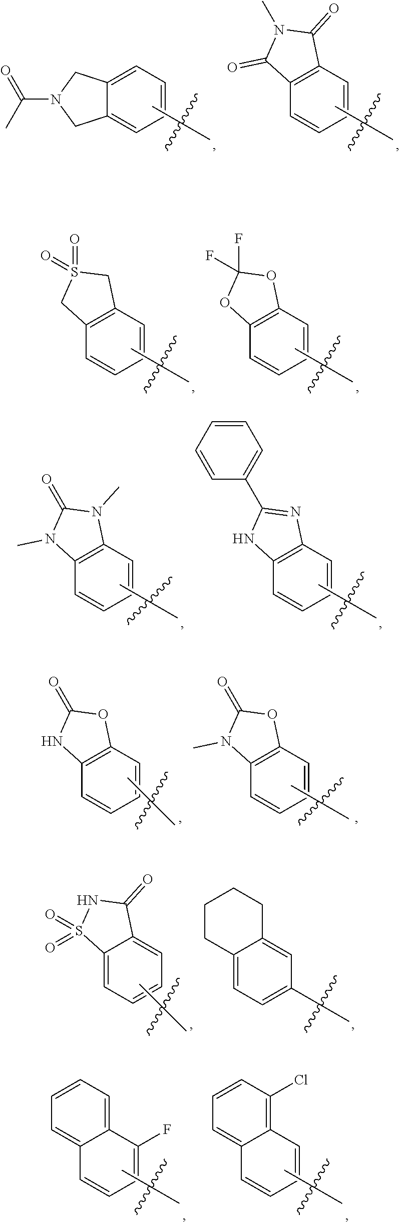

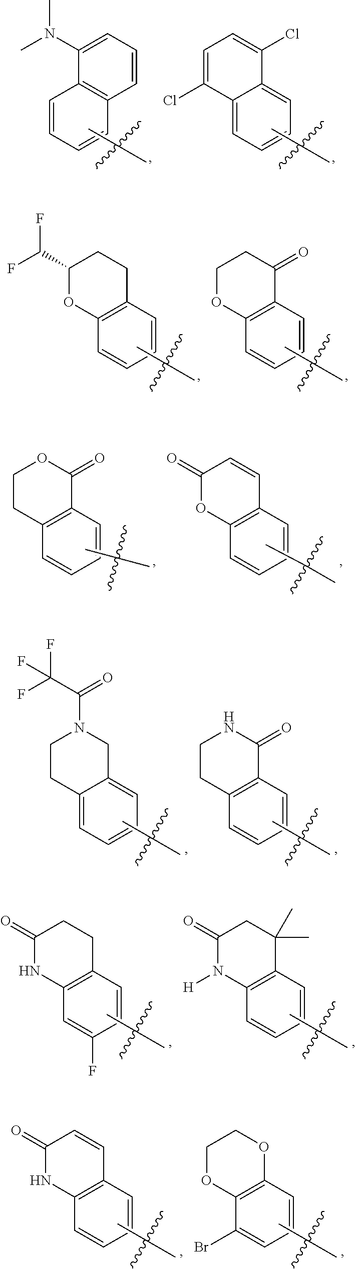

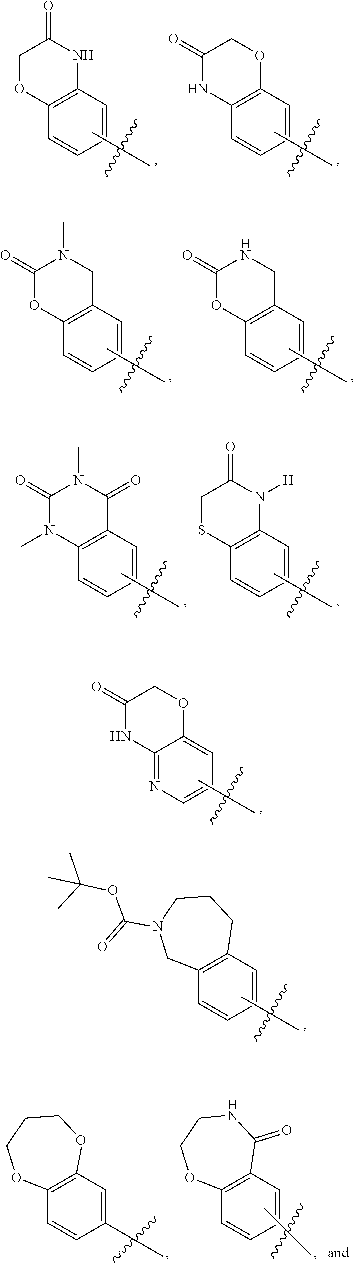

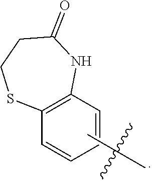

### Compounds of Formula II

In some embodiments the compound of Formula I is a compound of Formula II

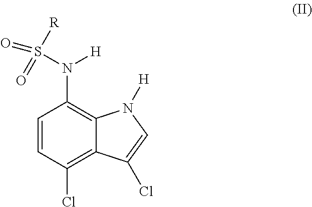

or a pharmaceutically acceptable salt thereof.

In some embodiments R of Formula II is selected from the group consisting of an optionally substituted -5,6; -6,5; -6,6; or -6,7 bicyclic ring system. In some embodiments R is a heteroaryl bicyclic ring or heterocyclic bicyclic ring. In some embodiments R of Formula II is any group described for substituent R of Formula I, described herein.

In some embodiments a compound of Formula II is a compound selected from:

| | |
|---|---|
| Compound 1 | 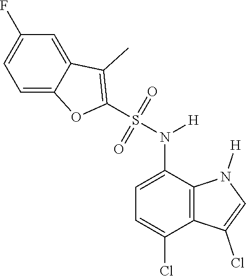 [SMILES: Cc1c(S(=O)(=O)Nc2ccc(Cl)c3c(Cl)c[nH]c23)oc2ccc(F)cc12] |
| Compound 2 | 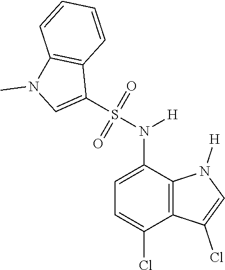 [SMILES: Cn1cc(S(=O)(=O)Nc2ccc(Cl)c3c(Cl)c[nH]c23)c2ccccc21] |
| Compound 3 | 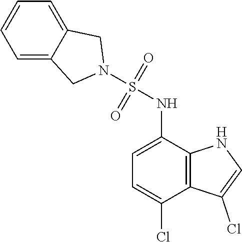 [SMILES: O=S(=O)(Nc1ccc(Cl)c2c(Cl)c[nH]c12)N1Cc2ccccc2C1] |
| Compound 4 | 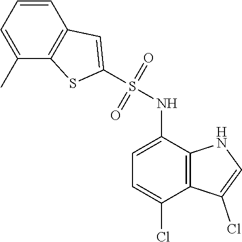 [SMILES: Cc1cccc2cc(S(=O)(=O)Nc3ccc(Cl)c4c(Cl)c[nH]c34)sc12] |
| Compound 5 | 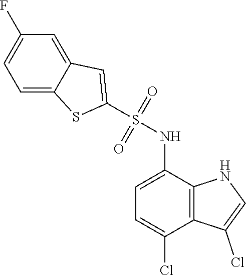 [SMILES: O=S(=O)(Nc1ccc(Cl)c2c(Cl)c[nH]c12)c1cc2cc(F)ccc2s1] |
| Compound 6 | 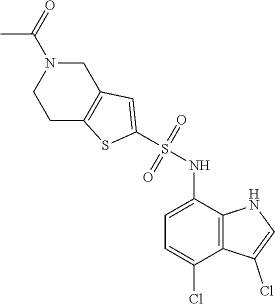 [SMILES: CC(=O)N1CCc2sc(S(=O)(=O)Nc3ccc(Cl)c4c(Cl)c[nH]c34)cc2C1] |
| Compound 7 | 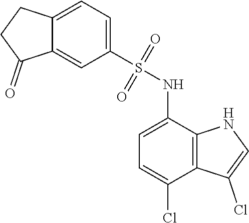 [SMILES: O=C1CCc2ccc(S(=O)(=O)Nc3ccc(Cl)c4c(Cl)c[nH]c34)cc21] |
| Compound 8 | 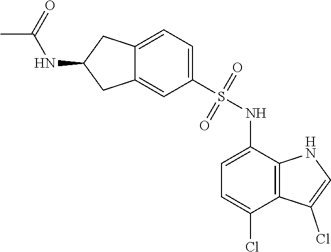 [SMILES: CC(=O)N[C@H]1Cc2ccc(S(=O)(=O)Nc3ccc(Cl)c4c(Cl)c[nH]c34)cc2C1] |
| Compound 9 | 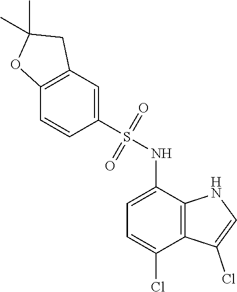 [SMILES: CC1(C)Cc2cc(S(=O)(=O)Nc3ccc(Cl)c4c(Cl)c[nH]c34)ccc2O1] |
| Compound 10 | 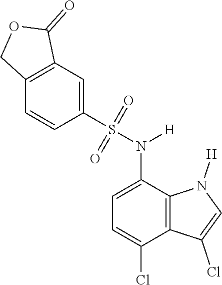 [SMILES: O=C1OCc2ccc(S(=O)(=O)Nc3ccc(Cl)c4c(Cl)c[nH]c34)cc21] |
| Compound 11 | 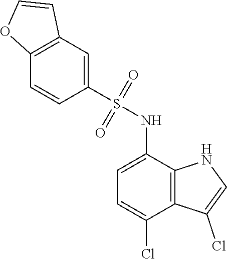 [SMILES: O=S(=O)(Nc1ccc(Cl)c2c(Cl)c[nH]c12)c1ccc2occc2c1] |
| Compound 12 | 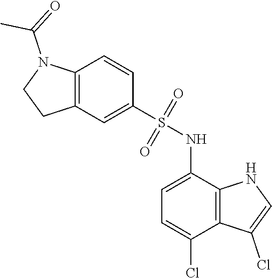 [SMILES: CC(=O)N1CCc2cc(S(=O)(=O)Nc3ccc(Cl)c4c(Cl)c[nH]c34)ccc21] |
| Compound 13 | 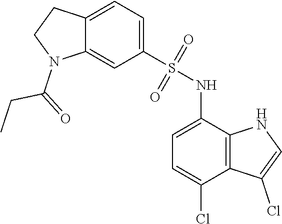 [SMILES: CCC(=O)N1CCc2ccc(S(=O)(=O)Nc3ccc(Cl)c4c(Cl)c[nH]c34)cc21] |
| Compound 14 | 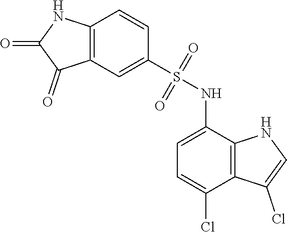 [SMILES: O=C1Nc2ccc(S(=O)(=O)Nc3ccc(Cl)c4c(Cl)c[nH]c34)cc2C1=O] |
| Compound 15 | 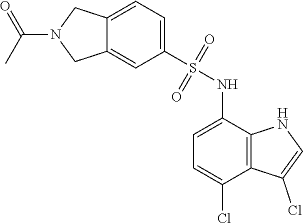 [SMILES: CC(=O)N1Cc2ccc(S(=O)(=O)Nc3ccc(Cl)c4c(Cl)c[nH]c34)cc2C1] |
| Compound 16 | 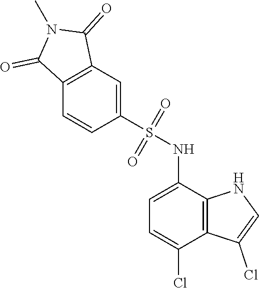 [SMILES: CN1C(=O)c2ccc(S(=O)(=O)Nc3ccc(Cl)c4c(Cl)c[nH]c34)cc2C1=O] |
| Compound 17 | 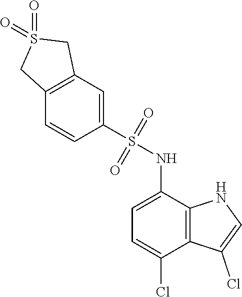 [SMILES: O=S1(=O)Cc2ccc(S(=O)(=O)Nc3ccc(Cl)c4c(Cl)c[nH]c34)cc2C1] |
| Compound 18 | 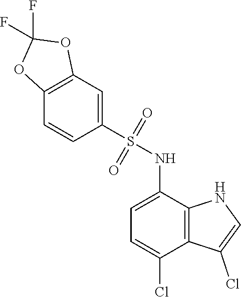 [SMILES: O=S(=O)(Nc1ccc(Cl)c2c(Cl)c[nH]c12)c1ccc2c(c1)OC(F)(F)O2] |
| Compound 19 | 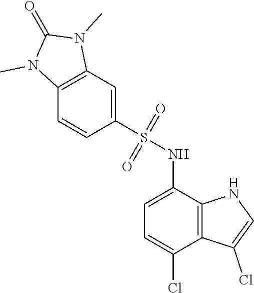 [SMILES: Cn1c(=O)n(C)c2cc(S(=O)(=O)Nc3ccc(Cl)c4c(Cl)c[nH]c34)ccc21] |
| Compound 20 | 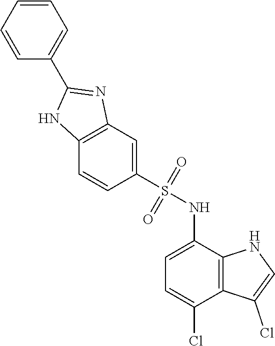 [SMILES: O=S(=O)(Nc1ccc(Cl)c2c(Cl)c[nH]c12)c1ccc2[nH]c(-c3ccccc3)nc2c1] |
| Compound 21 | 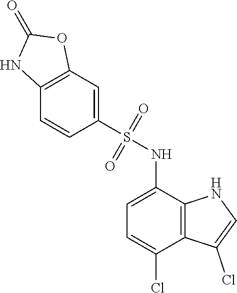 [SMILES: O=c1[nH]c2ccc(S(=O)(=O)Nc3ccc(Cl)c4c(Cl)c[nH]c34)cc2o1] |
| Compound 22 | 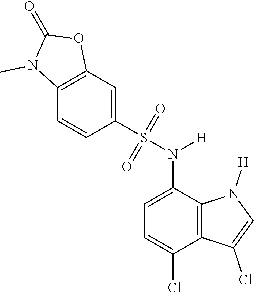 [SMILES: Cn1c(=O)oc2cc(S(=O)(=O)Nc3ccc(Cl)c4c(Cl)c[nH]c34)ccc21] |
| Compound 23 | 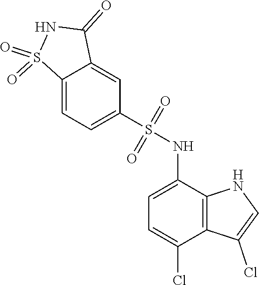 [SMILES: O=C1NS(=O)(=O)c2ccc(S(=O)(=O)Nc3ccc(Cl)c4c(Cl)c[nH]c34)cc21] |
| Compound 24 | 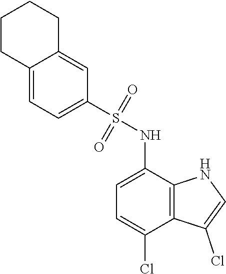 [SMILES: O=S(=O)(Nc1ccc(Cl)c2c(Cl)c[nH]c12)c1ccc2c(c1)CCCC2] |
| Compound 25 | 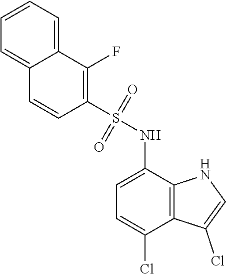 [SMILES: O=S(=O)(Nc1ccc(Cl)c2c(Cl)c[nH]c12)c1ccc2ccccc2c1F] |
| Compound 26 | 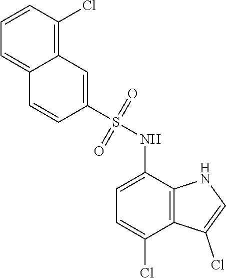 [SMILES: O=S(=O)(Nc1ccc(Cl)c2c(Cl)c[nH]c12)c1ccc2cccc(Cl)c2c1] |
| Compound 27 | 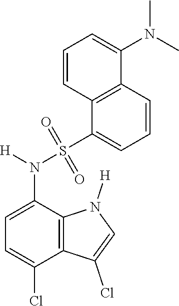 [SMILES: CN(C)c1cccc2c(S(=O)(=O)Nc3ccc(Cl)c4c(Cl)c[nH]c34)cccc12] |
| Compound 28 | 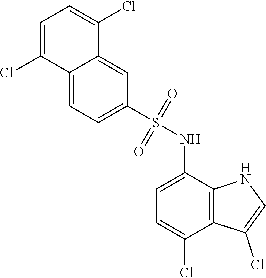 [SMILES: O=S(=O)(Nc1ccc(Cl)c2c(Cl)c[nH]c12)c1ccc2c(Cl)ccc(Cl)c2c1] |
| Compound 29 | 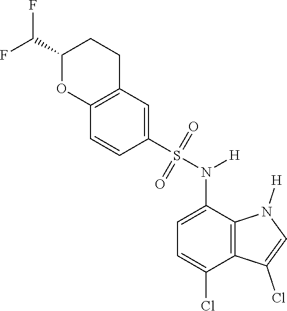 [SMILES: O=S(=O)(Nc1ccc(Cl)c2c(Cl)c[nH]c12)c1ccc2c(c1)CC[C@@H](C(F)F)O2] |
| Compound 30 | 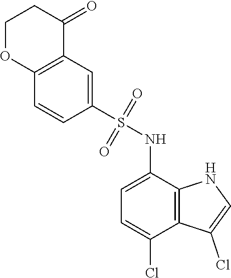 [SMILES: O=C1CCOc2ccc(S(=O)(=O)Nc3ccc(Cl)c4c(Cl)c[nH]c34)cc21] |
| Compound 31 | 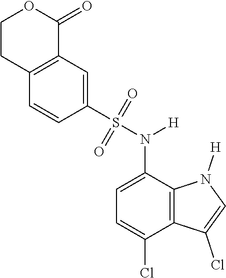 [SMILES: O=C1OCCc2ccc(S(=O)(=O)Nc3ccc(Cl)c4c(Cl)c[nH]c34)cc21] |
| Compound 32 | 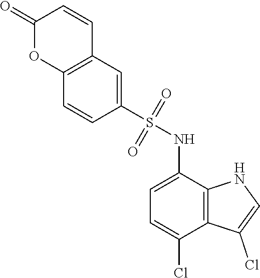 [SMILES: O=c1ccc2cc(S(=O)(=O)Nc3ccc(Cl)c4c(Cl)c[nH]c34)ccc2o1] |
| Compound 33 | 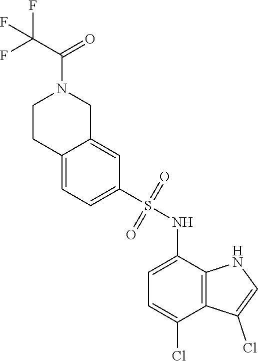 [SMILES: O=C(N1CCc2ccc(S(=O)(=O)Nc3ccc(Cl)c4c(Cl)c[nH]c34)cc2C1)C(F)(F)F] |
| Compound 34 | 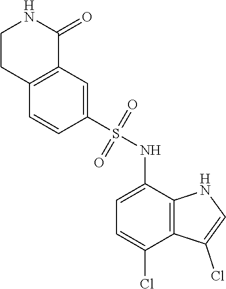 [SMILES: O=C1NCCc2ccc(S(=O)(=O)Nc3ccc(Cl)c4c(Cl)c[nH]c34)cc21] |
| Compound 35 | 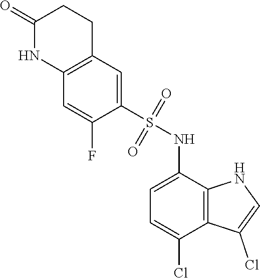 [SMILES: O=C1CCc2cc(S(=O)(=O)Nc3ccc(Cl)c4c(Cl)c[nH]c34)c(F)cc2N1] |
| Compound 36 | 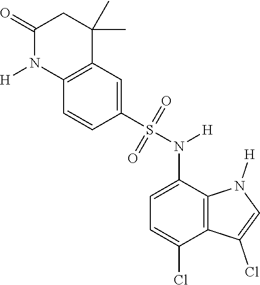 [SMILES: CC1(C)CC(=O)Nc2ccc(S(=O)(=O)Nc3ccc(Cl)c4c(Cl)c[nH]c34)cc21] |
| Compound 37 | 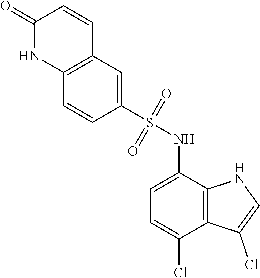 [SMILES: O=c1ccc2cc(S(=O)(=O)Nc3ccc(Cl)c4c(Cl)c[nH]c34)ccc2[nH]1] |
| Compound 38 | 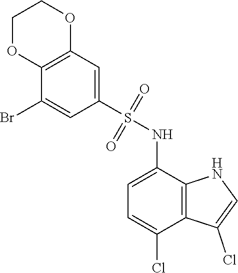 [SMILES: O=S(=O)(Nc1ccc(Cl)c2c(Cl)c[nH]c12)c1cc(Br)c2c(c1)OCCO2] |
| Compound 39 | 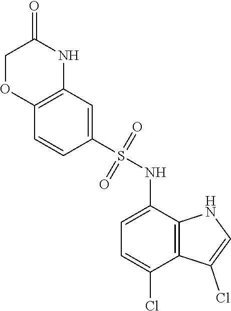 [SMILES: O=C1COc2ccc(S(=O)(=O)Nc3ccc(Cl)c4c(Cl)c[nH]c34)cc2N1] |
| Compound 40 | 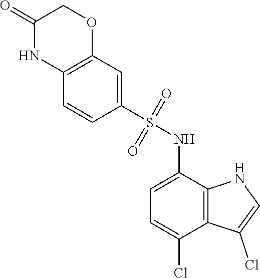 [SMILES: O=C1COc2cc(S(=O)(=O)Nc3ccc(Cl)c4c(Cl)c[nH]c34)ccc2N1] |
| Compound 41 | 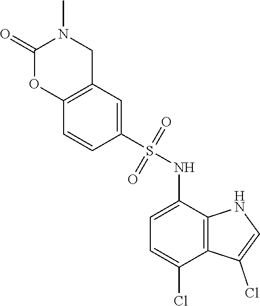 [SMILES: CN1Cc2cc(S(=O)(=O)Nc3ccc(Cl)c4c(Cl)c[nH]c34)ccc2OC1=O] |
| Compound 42 | 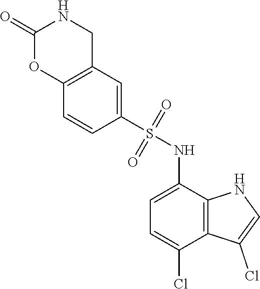 [SMILES: O=C1NCc2cc(S(=O)(=O)Nc3ccc(Cl)c4c(Cl)c[nH]c34)ccc2O1] |
| Compound 43 | 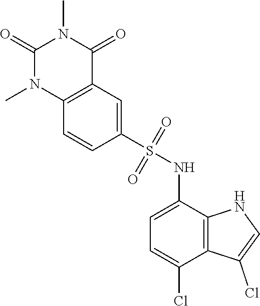 [SMILES: Cn1c(=O)c2cc(S(=O)(=O)Nc3ccc(Cl)c4c(Cl)c[nH]c34)ccc2n(C)c1=O] |
| Compound 44 | 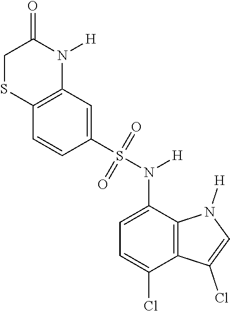 [SMILES: O=C1CSc2ccc(S(=O)(=O)Nc3ccc(Cl)c4c(Cl)c[nH]c34)cc2N1] |
| Compound 45 | 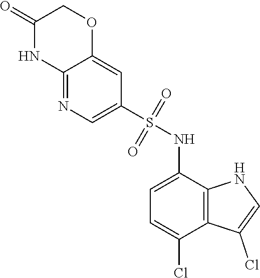 [SMILES: O=C1COc2cc(S(=O)(=O)Nc3ccc(Cl)c4c(Cl)c[nH]c34)cnc2N1] |
| Compound 46 | 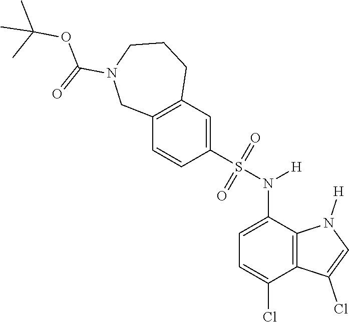 [SMILES: CC(C)(C)OC(=O)N1CCCc2cc(S(=O)(=O)Nc3ccc(Cl)c4c(Cl)c[nH]c34)ccc2C1] |
| Compound 47 | 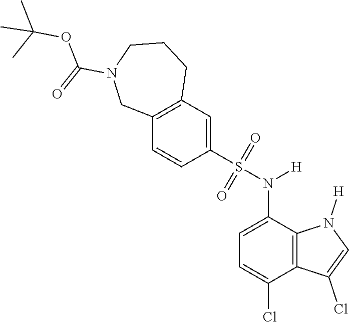 [SMILES: CC(C)(C)OC(=O)N1CCCc2cc(S(=O)(=O)Nc3ccc(Cl)c4c(Cl)c[nH]c34)ccc2C1] |
| Compound 48 | 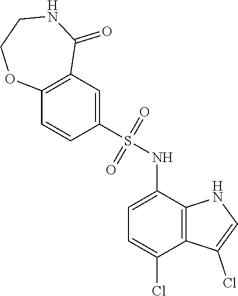 [SMILES: O=C1NCCOc2ccc(S(=O)(=O)Nc3ccc(Cl)c4c(Cl)c[nH]c34)cc21] and |
| Compound 49 | 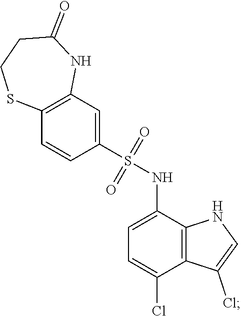 [SMILES: O=C1CCSc2ccc(S(=O)(=O)Nc3ccc(Cl)c4c(Cl)c[nH]c34)cc2N1] |

or a pharmaceutically acceptable salt thereof.

Some compounds of embodiments described herein can possess one or more asymmetric carbon atoms and are thus capable of existing in the form of optical isomers as well as in the form of racemic or non-racemic mixtures thereof. The compounds can be utilized in embodiments described herein as a single isomer or as a mixture of stereochemical isomeric forms. Diastereoisomers, i.e., non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of the invention can likewise be obtained by utilizing optically active starting materials. These isomers may be in the form of a free acid, a free base, an ester or a salt.

Compounds according to embodiments described herein may be in the form of pharmaceutically acceptable salts. A pharmaceutically acceptable salt of the compounds described herein includes acid addition salts and base addition salts. Pharmaceutically-acceptable salt embraces salts commonly used to form alkali metal salts and to form addition salts of free acids or free bases The nature of the salt is not critical, provided that it is pharmaceutically-acceptable. Suitable pharmaceutically-acceptable acid addition salts of the compounds described herein may be prepared from an inorganic acid or an organic acid. Examples of such inorganic acids include, without limitation, hydrochloric, hydrobromic, hydroiodic, nitric, carbonic, sulfuric and phosphoric acid. In some embodiments the salt is a hydrochloride salt. Appropriate organic acids may be selected from aliphatic, cycloaliphatic, aromatic, arylaliphatic, heterocyclic, carboxylic and sulfonic classes of organic acids, examples of which include, without limitation, formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically-acceptable base addition salts for compounds described herein can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cyclo alkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the preparation of pharmaceutically acceptable salts, for example, carboxylic acid amides, including carboxamides, lower alkyl carboxamides, dialkyl carboxamides, and the like. Acceptable salts may be obtained using standard procedures well known in the art, for example by treating a sufficiently basic compound such as an amine with a suitable acid affording a physiologically acceptable anion. Alkali metal (for example, sodium, potassium or lithium) or alkaline earth metal (for example calcium) salts of organic (e.g., carboxylic) acids can also be made.

The compounds disclosed herein can exist as and therefore include all tautomers, and mixtures thereof in all proportions.

### Pharmaceutical Compositions

Some embodiments describe a pharmaceutical composition comprising: a compound according to any embodiment described herein, or a pharmaceutically acceptable salt thereof; and a pharmaceutically acceptable carrier or diluent. The pharmaceutical compositions can be prepared in a manner well known in the pharmaceutical art, and can be administered by a variety of routes, depending upon whether local or systemic treatment is desired and upon the area to be treated.

While it is possible that a compound as described in any embodiment herein, may be administered as the bulk substance, it is preferable to present the compound in a pharmaceutical formulation, e.g., wherein the active agent is in an admixture with a pharmaceutically acceptable carrier selected with regard to the intended route of administration and standard pharmaceutical practice.

In particular, the disclosure provides a pharmaceutical composition comprising a therapeutically effective amount of at least one compound according to any embodiment described herein, and optionally, a pharmaceutically acceptable carrier.

For the pharmaceutical compositions and methods of the disclosure, a compound according to any embodiment described herein, may be used in combination with other therapies and/or active agents.

Accordingly, the disclosure provides, in a further aspect, a pharmaceutical composition comprising at least one compound according to any embodiment described herein, or pharmaceutically acceptable salt thereof; a second active agent; and, optionally a pharmaceutically acceptable carrier.

When used in combination therapy, the compounds described herein are administered with the second active agent simultaneously or separately. Thus the combination of the two agents can be administered simultaneously in the same dosage form, simultaneously in separate dosage forms or administered separately. When combined in the same formulation it will be appreciated that the two or more compounds must be stable and compatible with each other and the other components of the formulation. When formulated separately they may be provided in any convenient formulation, in such a manner as are known for such compounds in the art. In some embodiments, a compound according to any embodiment described herein is administered first, followed by administration of the second agent. In some embodiments the second agent is administered first, followed by administration of the first. In some embodiments the administration of the two agents can begin simultaneously, but is not completed at the same time. For example, one agent is administered orally, simultaneously with commencement of the administration of a second second agent via a thirty minute bolus IV. In some embodiments a compound according to embodiments described herein and a second active agent are administered separately a few minutes apart, a few hours apart or a few days apart.

Preservatives, stabilizers, dyes and flavoring agents may be provided in any pharmaceutical composition described herein. Examples of preservatives include sodium benzoate, ascorbic acid and esters of p-hydroxybenzoic acid. Antioxidants and suspending agents may be also used.

### Routes of Administration and Dosage Forms

Compounds according to any embodiment described herein and pharmaceutically acceptable salts thereof can be formulated for any route of administration.

The routes for administration (delivery) include, but are not limited to, one or more of: oral (e.g., as a tablet, capsule, or as an ingestible solution), topical, mucosal (e.g., as a nasal spray or aerosol for inhalation), parenteral (e.g., by an injectable form), gastrointestinal, intraspinal, intraperitoneal, intramuscular, intravenous, intracerebroventricular, or other depot administration etc.

Therefore, the pharmaceutical compositions according to any embodiment described herein, include those in a form especially formulated for the mode of administration. In certain embodiments, the pharmaceutical compositions of the disclosure are formulated in a form that is suitable for oral delivery. In some embodiments, the compound is an orally bioavailable compound, suitable for oral delivery. In other embodiments, the pharmaceutical compositions of the disclosure are formulated in a form that is suitable for parenteral delivery.

The compounds according to any embodiment described herein, may be formulated for administration in any convenient way for use in human or veterinary medicine and the disclosure therefore includes within its scope pharmaceutical compositions comprising a compound according to any embodiment described herein, adapted for use in human or veterinary medicine. Such pharmaceutical compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

There may be different pharmaceutical composition/formulation requirements depending on the different delivery systems. It is to be understood that not all of the compounds need to be administered by the same route. Likewise, if the pharmaceutical composition comprises more than one active component, then those components may be administered by different routes. By way of example, the pharmaceutical composition of the disclosure may be formulated to be delivered using a mini-pump or by a mucosal route, for example, as a nasal spray or aerosol for inhalation or ingestible solution, or parenterally in which the pharmaceutical composition is formulated by an injectable form, for delivery by, for example, an intravenous, intramuscular or subcutaneous route. Alternatively, the formulation may be designed to be delivered by multiple routes.

Where appropriate, the pharmaceutical compositions according to any embodiment described herein, can be administered by inhalation, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the pharmaceutical compositions according to any embodiment described herein, may be administered in the form of tablets or lozenges, which can be formulated in a conventional manner.

Where the pharmaceutical composition according to any embodiment described herein, is to be administered parenterally, such administration includes without limitation: intravenously, intraarterially, intrathecally, intraventricularly, intracranially, intramuscularly or subcutaneously administering the compound of the disclosure; and/or by using infusion techniques.

Pharmaceutical compositions according to any embodiment described herein, suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. In all cases, the final preparation must be sterile, liquid, and stable under production and storage conditions. To improve storage stability, such preparations may also contain a preservative to prevent the growth of microorganisms. Prevention of the action of micro-organisms can be achieved by the addition of various antibacterial and antifungal agents, e.g., paraben, chlorobutanol, or ascorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminum monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound according to any embodiment described herein, is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound according to any embodiment described herein, with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the compounds and desired excipients for subsequent preparation of sterile solutions.

The compounds according to any embodiment described herein, may be formulated for use in human or veterinary medicine by injection (e.g., by intravenous bolus injection or infusion or via intramuscular, subcutaneous or intrathecal routes) and may be presented in unit dose form, in ampoules, or other unit-dose containers, or in multi-dose containers, if necessary with an added preservative. The pharmaceutical compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds according to any embodiment described herein, can be administered in the form of tablets, capsules, troches, ovules, elixirs, solutions or suspensions, for immediate-, delayed-, modified-, sustained-, pulsed- or controlled-release applications.

The compounds according to any embodiment described herein, may also be presented for human or veterinary use in a form suitable for oral or buccal administration, for example in the form of solutions, gels, syrups, or suspensions, or a dry powder for reconstitution with water or other suitable vehicle before use. Solid pharmaceutical compositions such as tablets, capsules, lozenges, troches, pastilles, pills, boluses, powder, pastes, granules, bullets or premix preparations may also be used. Solid and liquid pharmaceutical compositions for oral use may be prepared according to methods well-known in the art. Such pharmaceutical compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The pharmaceutical compositions according to any embodiment described herein, may be administered orally, in the form of rapid or controlled release tablets, microparticles, mini tablets, capsules, sachets, and oral solutions or suspensions, or powders for the preparation thereof. Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the pharmaceutical compositions, e.g., act as both binders and disintegrants.

Examples of pharmaceutically acceptable disintegrants for oral pharmaceutical compositions according to any embodiment described herein, include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and cross-linked polyvinylpyrrolidone.

Examples of pharmaceutically acceptable binders for oral pharmaceutical compositions according to any embodiment described herein, include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthine resin, alginates, magnesium aluminum silicate, polyethylene glycol or bentonite.

Examples of pharmaceutically acceptable fillers for oral pharmaceutical compositions according to any embodiment described herein, include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulphate.

Examples of pharmaceutically acceptable lubricants useful in the pharmaceutical compositions according to any embodiment described herein, include, but are not limited to, magnesium stearate, talc, polyethylene glycol, polymers of ethylene oxide, sodium lauryl sulphate, magnesium lauryl sulphate, sodium oleate, sodium stearyl fumarate, and colloidal silicon dioxide.

Examples of suitable pharmaceutically acceptable odorants for the oral pharmaceutical compositions according to any embodiment described herein, include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral pharmaceutical compositions according to any embodiment described herein, include, but are not limited to, synthetic and natural dyes such as titanium dioxide, beta-carotene and extracts of grapefruit peel.

Examples of useful pharmaceutically acceptable coatings for the oral pharmaceutical compositions according to any embodiment described herein, typically used to facilitate swallowing, modify the release properties, improve the appearance, and/or mask the taste of the pharmaceutical compositions include, but are not limited to, hydroxypropylmethylcellulose, hydroxypropylcellulose and acrylate-methacrylate copolymers.

Suitable examples of pharmaceutically acceptable sweeteners for the oral pharmaceutical compositions according to any embodiment described herein, include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulphate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

As indicated, a compounds according to any embodiment described herein, can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of a compound according to any embodiment described herein, and a suitable powder base such as lactose or starch.

For topical administration by inhalation a compounds according to any embodiment described herein, may be delivered for use in human or veterinary medicine via a nebulizer.

The pharmaceutical compositions of the disclosure may contain from 0.01 to 99% weight per volume of the active material. For topical administration, for example, the pharmaceutical composition will generally contain from 0.01-10%, more preferably 0.01-1% of the active material.

A compound according to any embodiment described herein, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage form, according to any embodiment described herein, may be administered according to a dosage and administration regimen defined by routine testing in the light of the guidelines given above in order to obtain optimal activity while minimizing toxicity or side effects for a particular patient. The dosage of the compounds or unit dosage form may vary according to a variety of factors such as underlying disease conditions, the individual's condition, weight, sex and age, and the mode of administration. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter is within the scope of the disclosure. Clinically significant attenuation or amelioration means perceptible to the patient and/or to the physician.

A pharmaceutical composition for parenteral administration contains from about 0.01% to about 100% by weight of the active compound according to any embodiment described herein, based upon 100% weight of total pharmaceutical composition.

Generally, transdermal dosage forms contain from about 0.01% to about 100% by weight of the active compound according to any embodiment described herein, versus 100% total weight of the dosage form.

The pharmaceutical composition or unit dosage form may be administered in a single daily dose, or the total daily dosage may be administered in divided doses. In addition, co administration or sequential administration of another compound for the treatment of the disorder may be desirable. To this purpose, the combined active principles are formulated into a simple dosage unit.

### Methods of Use

In some embodiments, the present disclosure provides a method of inducing degradation of a protein comprising contacting the protein with an effective amount of a compound or composition according to any embodiment described herein.

Without wishing to be bound by any particular theory, the inventors believe that a compound according to any embodiment herein, can act as a molecular glue and induce an interaction between a ubiquitin E3 ligase and a target protein, leading to degradation of the target protein. Inducing protein degradation results in a decrease in protein levels. In some embodiments, the compounds promotes the formation of a complex between the protein and a substrate recognition subunit of the E3 ligase. In some instances a compound according to any embodiment herein binds to the ubiquitin E3 ligase and recruits proteins for degradation by the ubiquitin-proteasome system. In some embodiments the substrate recognition subunit of the E3 ligase is DCAF15. In some embodiments the substrate recognition subunit of the E3 ligase is human DCAF15. An example of human DCAF15 is Uniprot ID: Q66K64. In some embodiments the compound binds to the protein. In some embodiments the protein is a protein that may lack enzymatic active sites that can be targeted by inhibitors. In some embodiments the protein is RBM39. In some embodiments the substrate recognition subunit of the E3 ligase is DCAF15 and the protein is RBM39.

Some embodiments describes a method of inducing degradation of a protein, in a patient in need thereof, comprising administering to the patient, a therapeutically effective amount of a compound according to any embodiment described herein.

In some embodiments the compounds and compositions according to embodiments of the present invention are useful for treating a disease, disorder or condition associated with a target protein. Any disease, disorder or condition that results directly or indirectly from abnormal activity of the target protein or expression level of the gene that encodes the target protein can be an intended disease condition. Some embodiments describe a method of treating a disease or disorder that results from abnormal activity of a target protein in a subject, comprising administering to the subject, a therapeutically effective amount of a compound according to any embodiment described herin, wherein the compound induces degradation of the protein thereby treating the disease or disorder. In some embodiments the disease or disorder results directly from abnormal activity of a target protein. In some embodiments the disease or disorder results indirectly from the abnormal activity of the target protein. Some embodiments describe a method of treating a disease or disorder that results from abnormal expression of a gene that encodes a target protein in a subject, comprising administering to the subject, a therapeutically effective amount of a compound according to any any embodiment described herein, wherein the compound induces degradation of the protein thereby treating the disease or disorder.

Some embodiments of the invention describe use of a compound according to any any embodiment described herein, in the preparation of a medicament for promoting the degradation of a protein, in a patient in need thereof. Some embodiments describe a compound according to any embodiment described herein for use in medical therapy.

A number of assay systems can be used to determine if the compounds according to embodiments describe herein, are useful in the methods described above. For example, assays that measure if the compound induces the formation of a complex between the E3-ligase subunit and the target protein; assays that measure if the compound binds the the E-3 ligase subunit, assays that measure if protein expression is decreased in a cell upon addition of the compounds and assays that measure cell viability. A non-limiting list of assays includes amplified luminescent proximity homogenous assay, homogeneous time fluorescence competition assays, Western blot assay, and CellTiter-Glo® Luminescent Cell Viability Assay.

These assays can be conducted with one or more positive control compounds-compounds that are known to induce an interaction between a ubiquitin E3 ligase and a target protein, leading to the degradation of the target protein. Examples of such positive control compounds, includes indisulam, E-7820; tasisulam and cloroquinoxaline sulfonamide (CQS).

Compounds of the invention were tested in the assays described in Examples 105-108.

### EXAMPLES

The following examples are given to illustrate the present subject matter. It should be understood, however, that the subject matter is not limited to the specific conditions or details described in these examples.

### Preparative HPLC Condition: Basic Method

Instrument: Shimadzu LCMS 2010A
Column: Phenomenex Gemin-NX 150*30 mm*5 um, Boston Prime C18 150*30 mm*5 um, Agela Durashell C18 150×25 mm*5 um, Xtimate C18 150*25 mm*5 um
Mobile phase A: 10 mM NH4HCO3 and 0.01% NH4OH in water
Mobile phase B: Acetonitrile
Gradient: 80% A/20% B linear to 60% A/40% B in 10.0 min, hold at 0% A/100% B to 12.0 min.
Flow rate: 35 mL/min or 30 mL/min

### Preparative TLC Condition:

TLC plate: 20*20 cm glass plate,
Spread solvent: EtOAc/PE; MeOH/EtOAc

### QC Method:

Method NEGCD05-220

| Column Xbridge C18 | 2.1 × 50 mm 5 μm | |
|---|---|---|
| Temperature | 40° | C. |
| Mobile Phase A | 0.05% NH4OH in water | |
| Mobile Phase B | 100% acetonitrile | |
| Time 0.00 mins | 5% B | |
| Time 3.60 mins | 100% B | |
| Time 3.90 mins | 100% B | |
| Time 3.91 mins | 5% B | |
| Time 4.50 mins | stop | |
| Flow rate | 0.8 | ml/min |
| wavelength | 220 | nm |
| Injection volume | 2 | μl |

| | |
|---|---|
| Agilent 1200 HPLC/6100Series | MSD |
| Ionization Mode | API-ES |
| Polarity | Negative |

### General Synthetic Method:

The compounds of the present invention can be prepared according to the following general scheme:

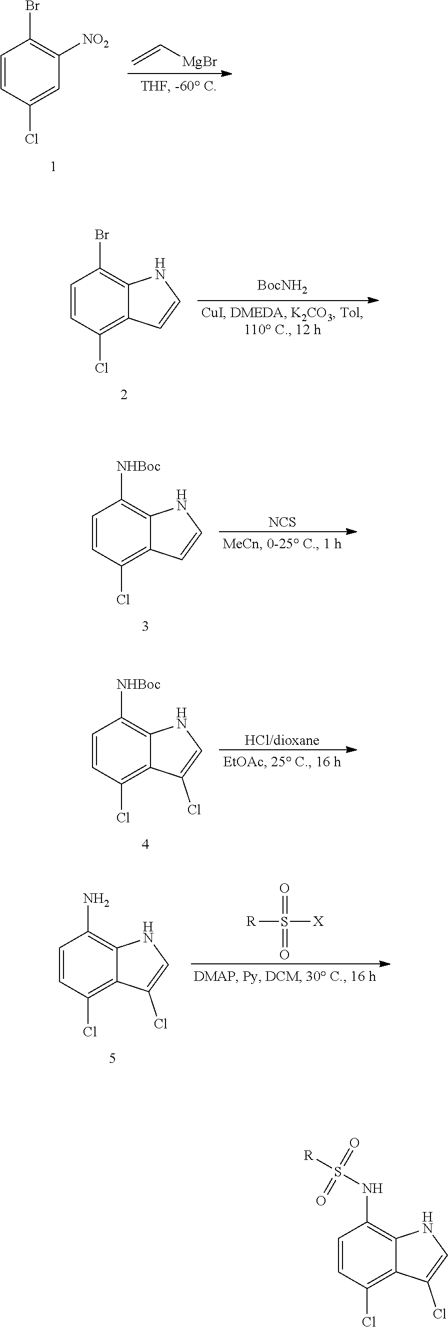

### Example 1: Preparation of Intermediate 5

### Preparation of Intermediate 2

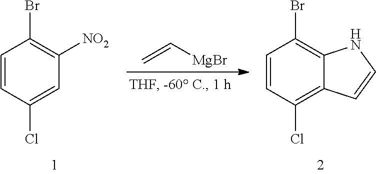

To a solution of 1 (100 g, 422.92 mmol, 1 eq) in dry THF (1.2 L) was added bromo(vinyl)magnesium (1.6 M, 1.32 L, 5 eq) at −60° C. under N2. The reaction mixture was stirred at −60° C. for 1 h. The reaction mixture was poured into aqueous NH₄Cl (1000 mL) and extracted with EtOAc (3000 mL×3). The combined organic layer was washed with brine, dried over anhydrous Na₂SO₄, filtered, concentrated in vacuo to give a crude product. The residue was purified by flash silica gel chromatography (ISCO®; 330 g SepaFlash® Silica Flash Column, Eluent of 0-5% Ethyl acetate/Petroleum ethergradient @100 mL/min). Intermediate 2 (40 g, 121.48 mmol, 28.72% yield, 70% purity) was obtained as a yellow oil.

¹H NMR (400 MHz, DMSO-d6) δ ppm 11.74 (br s, 1H) 7.53 (t, J=2.76 Hz, 1H) 7.32 (d, J=8.03 Hz, 1H) 7.04 (d, J=8.03 Hz, 1H) 6.60 (dd, J=2.89, 2.13 Hz, 1H).

### Preparation of Intermediate 3

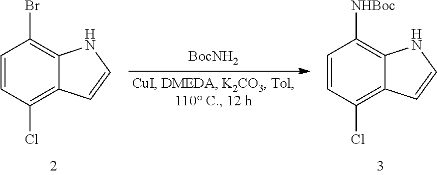 [SMILES: Clc1ccc(Br)c2[nH]ccc12 ; "BocNH2" ; CC(C)(C)OC(=O)Nc1ccc(Cl)c2cc[nH]c12]

To a mixture of intermediate 2 (36 g, 109.33 mmol, 70% purity, 1 eq), tert-butyl carbamate (14.09 g, 120.27 mmol, 1.1 eq), K₂CO₃ (30.22 g, 218.67 mmol, 2 eq), DMEDA (963.79 mg, 10.93 mmol, 1.18 mL, 0.1 eq) in toluene (300 mL) was added CuI (1.04 g, 5.47 mmol, 0.05 eq) under N2, then the mixture was stirred at 110° C. for 12 h. The reaction mixture was filtered and concentrated under reduced pressure to remove solvent. The residue was purified by flash silica gel chromatography (ISCO®; 330 g SepaFlash® Silica Flash Column, Eluent of 0-20% Ethyl acetate/Petroleum ethergradient @ 100 mL/min). Intermediate 3 (4.8 g, 17.10 mmol, 15.64% yield, 95% purity) was obtained as a yellow solid.

¹H NMR (400 MHz, DMSO-d6) δ ppm 11.14 (br s, 1H) 9.11 (br s, 1H) 7.36-7.58 (m, 2H) 6.88-7.11 (m, 1H) 6.45 (br s, 1H) 1.51 (s, 9H)

### Preparation of Intermediate 4

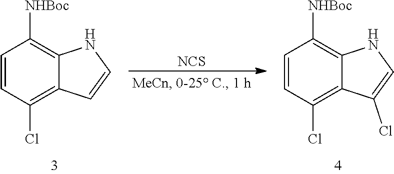 [SMILES: CC(C)(C)OC(=O)Nc1ccc(Cl)c2cc[nH]c12 ; "NCS" ; CC(C)(C)OC(=O)Nc1ccc(Cl)c2c(Cl)c[nH]c12]

To a solution of NCS (2.63 g, 19.68 mmol, 1.05 eq) in MeCN (50 mL) was added intermediate 3 (5.0 g, 18.75 mmol, 1 eq) in MeCN (100 mL) at 0° C. and the reaction was stirred at 25° C. for 2 h. The solution was clear. Saturated Na₂SO₃ (20 mL) was added to the reaction. The organic solvent was removed under reduced pressure. The residue was diluted with water (100 mL) and extracted with EtOAc (100 mL×3). The combined organic layer was washed with brine, dried over anhydrous Na₂SO₄, filtered and concentrated to give a crude product. The residue was purified by flash silica gel chromatography (ISCO®; 120 g SepaFlash® Silica Flash Column, Eluent of 0-20% Ethylacetate/Petroleum ethergradient @100 mL/min). Compound 4 (4.4 g, 14.61 mmol, 77.94% yield, 100% purity) was obtained as a yellow solid.

¹H NMR (400 MHz, DMSO-d6) δ ppm 11.40 (br s, 1H) 9.09 (br s, 1H) 7.60 (d, J=2.76 Hz, 1H) 7.49 (br d, J=7.03 Hz, 1H) 7.02 (d, J=8.53 Hz, 1H) 1.50 (s, 9H)

### Preparation of Intermediate 5

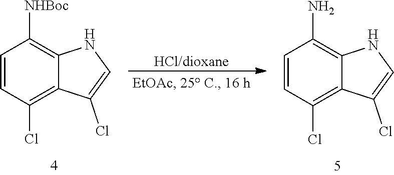 [SMILES: CC(C)(C)OC(=O)Nc1ccc(Cl)c2c(Cl)c[nH]c12 ; "HCl/dioxane" ; Nc1ccc(Cl)c2c(Cl)c[nH]c12]

To a mixture of intermediate 4 (4.4 g, 14.61 mmol, 1 eq) in EtOAc (50 mL) was added HCl/dioxane (4 M, 39.11 mL, 10.71 eq), then the mixture was stirred at 25° C. for 16 h. Yellow solid was formed. The solid was filtered and concentrated. The product was used for the next step without purification. Intermediate 5 (3.25 g, 13.27 mmol, 90.85% yield, 97% purity, HCl) was obtained as a yellow solid.

¹H NMR (400 MHz, DMSO-d6) δ ppm 11.96 (br s, 1H) 7.71 (d, J=2.76 Hz, 1H) 7.05 (d, J=8.03 Hz, 1H) 6.92 (br d, J=8.28 Hz, 1H).

### Example 2: General Procedure for Preparation of Compounds

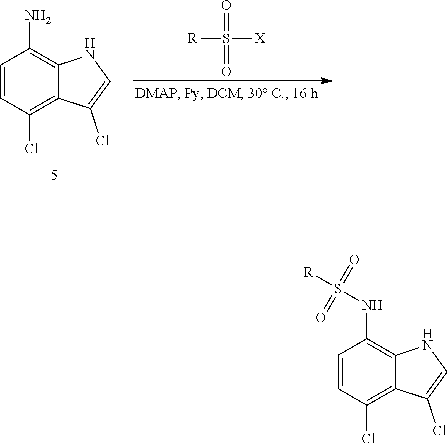

To a solution of intermediate 5 (150 umol, 1.0 eq.) in DCM (600 uL) was added a solution of sulfonyl chloride (150 umol, 1.0 eq.) in Py (600 μL). Then DMAP (15 umol, 0.1 eq) was dispensed to each vial. Vials were capped and shaken at 30° C. for 16 hrs. The solvent was removed by Speedvac and crude product was purified by preparative HPLC to give the final product.

### Example 3: Preparation of Compound 43: N-(3,4-dichloro-1H-indol-7-yl)-1,3-dimethyl-2,4-dioxo-1,2,3,4-tetrahydroquinazoline-6-sulfonamide

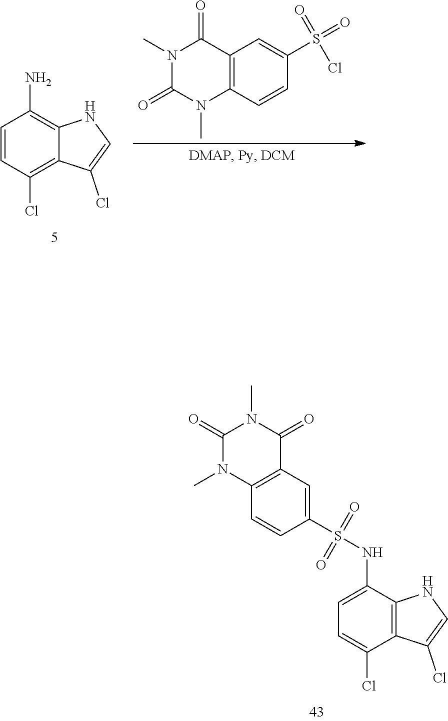 [SMILES: Nc1ccc(Cl)c2c(Cl)c[nH]c12 ; Cn1c(=O)c2cc(S(=O)(=O)Cl)ccc2n(C)c1=O ; Cn1c(=O)c2cc(S(=O)(=O)Nc3ccc(Cl)c4c(Cl)c[nH]c34)ccc2n(C)c1=O]

To a solution of 3,4-dichloro-1H-indol-7-amine (intermediate 5, 30.2 mg, 0.15 mmol) in DCM (0.6 mL), was added a solution of 1,3-dimethyl-2,4-dioxo-1,2,3,4-tetrahydroquinazoline-6-sulfonyl chloride (43.3 mg, 0.15 mmol) in Py (0.6 mL) in an 8 mL reaction vial. Then DMAP (1.8 mg, 0.015 mmol) was added to the vial. The vial was capped and shaken at 30° C. for 16 hrs. Solvent was removed by Speedvac and the crude product was purified by Basic preparative HPLC to give final product N-(3,4-dichloro-1H-indol-7-yl)-1,3-dimethyl-2,4-dioxo-1,2,3,4-tetrahydroquinazoline-6-sulfonamide (Compound 43, 35.48 mg, yield: 52.2%). LCMS (ESI) found: 565.0 [M-H]⁻.

¹H NMR (400 MHz, DMSO-d₆) δ ppm 11.49 (d, 1H) 10.12 (s, 1H) 8.38 (d, 1H) 7.95 (d, 1H) 7.51-7.61 (m, 2H) 6.94 (d, 1H) 6.69 (d, 1H) 3.50 (s, 1H) 2.54 (s, 3H).

### Example 4: Preparation of Compound 44: N-(3,4-dichloro-1H-indol-7-yl)-3-oxo-3,4-dihydro-2H-benzo[b][1,4]thiazine-6-sulfonamide

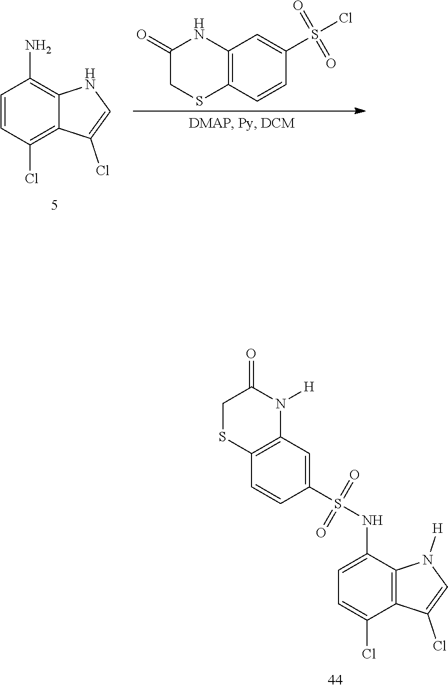 [SMILES: Nc1ccc(Cl)c2c(Cl)c[nH]c12 ; O=C1CSc2ccc(S(=O)(=O)Cl)cc2N1 ; O=C1CSc2ccc(S(=O)(=O)Nc3ccc(Cl)c4c(Cl)c[nH]c34)cc2N1]

To a solution of 3,4-dichloro-1H-indol-7-amine (intermediate 5, 30.2 mg, 0.15 mmol) in DCM (0.6 mL) was added a solution of 3-oxo-3,4-dihydro-2H-benzo[b][1,4]thiazine-6-sulfonyl chloride (39.6 mg, 0.15 mmol) in Py (0.6 mL) in an 8 mL reaction vial. DMAP (1.8 mg, 0.015 mmol) was added to the vial. The vial was capped and shaken at 30° C. for 16 hrs. Solvent was removed by Speedvac and the crude product was purified by Basic preparative HPLC to give final product N-(3,4-dichloro-1H-indol-7-yl)-3-oxo-3,4-dihydro-2H-benzo[b][1,4]thiazine-6-sulfonamide (Compound 44, 27.20 mg, yield: 42.4%). LCMS (ESI) found: 425.9 [M-H].

### Example 5: Preparation of Compound 42: N-(3,4-dichloro-1H-indol-7-yl)-2-oxo-3,4-dihydro-2H-benzo[e][1,3]oxazine-6-sulfonamide

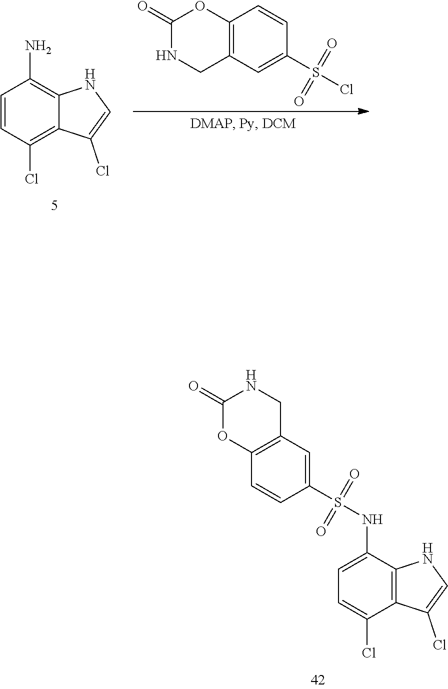 [SMILES: Nc1ccc(Cl)c2c(Cl)c[nH]c12 ; O=C1NCc2cc(S(=O)(=O)Cl)ccc2O1 ; O=C1NCc2cc(S(=O)(=O)Nc3ccc(Cl)c4c(Cl)c[nH]c34)ccc2O1]

To a solution of 3,4-dichloro-1H-indol-7-amine (intermediate 5, 30.2 mg, 0.15 mmol) in DCM (0.6 mL) was added a solution of 2-oxo-3,4-dihydro-2H-benzo[e][1,3]oxazine-6-sulfonyl chloride (37.2 mg, 0.15 mmol) in Py (0.6 mL) in 8 mL reaction vial. DMAP (1.8 mg, 0.015 mmol) was added to the vial. The vial was capped and shaken at 30° C. for 16 hrs. Solvent was removed by Speedvac and the crude product was purified by Basic preparative HPLC to give final product N-(3,4-dichloro-1H-indol-7-yl)-2-oxo-3,4-dihydro-2H-benzo[e][1,3]oxazine-6-sulfonamide (Compound 42, 19.11 mg, yield: 30.9%). LCMS (ESI) found: 409.9 [M-H].⁻

### Example 6: Preparation of Compound 17

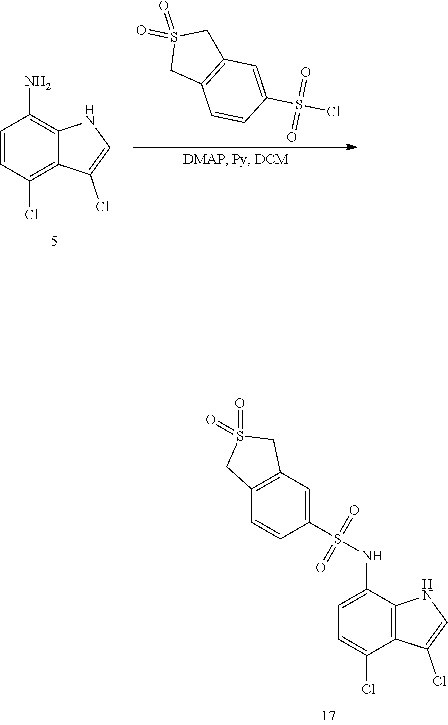 [SMILES: Nc1ccc(Cl)c2c(Cl)c[nH]c12 ; O=S1(=O)Cc2ccc(S(=O)(=O)Cl)cc2C1 ; O=S1(=O)Cc2ccc(S(=O)(=O)Nc3ccc(Cl)c4c(Cl)c[nH]c34)cc2C1]

To a solution of 3,4-dichloro-1H-indol-7-amine (intermediate 5, 30.2 mg, 0.15 mmol) in DCM (0.6 mL) was added a solution of 1,3-dihydrobenzo[c]thiophene-5-sulfonyl chloride 2,2-dioxide (40.0 mg, 0.15 mmol) in Py (0.6 mL) in an 8 mL reaction vial. DMAP (1.8 mg, 0.015 mmol) was added to the vial. The vial was capped and shaken at 30° C. for 16 hrs. Solvent was removed by Speedvac and the crude product was purified by Basic preparative HPLC to give final product N-(3,4-dichloro-1H-indol-7-yl)-1,3-dihydrobenzo[c]thiophene-5-sulfonamide 2,2-dioxide (Compound 17, 19.58 mg, yield: 30.3%). LCMS (ESI) found: 428.9 [M-H].⁻

### Example 7: Quantitative Evaluation of Compound-Induced Ternary Complex Formation Using Amplified Luminescent Proximity Homogeneous Assay

Compound-induced DCAF15-RBM39 ternary complex formation was monitored using AlphaLISA® assays (Perkin Elmer, Waltham, MA). Test compounds were first collected in a 384-well microplate (cat. #781280, Grenier Bio-One, Monroe, NC) as 2 mM stocks in 100% dimethyl sulfoxide (DMSO). These stocks were then serially diluted in DMSO by 3-fold into a new plate (cat. #781280, Grenier Bio-One, Monroe, NC) using a Mosquito HTS dispenser (Mosquito® HTS, SPT Labtech, Boston, MA). 0.2 μL of these diluted stocks were then dispensed into a 384-well Optiplate (cat. #6007290, Perkin Elmer, Waltham, MA) in duplicate wells, to assemble the final assay plate consisting of duplicated dose ranges. Separately, a protein mixture consisting of His-tagged DCAF15 complex and biotin-carrying RBM39, each at 408 nM (2.04×), was prepared in 50 mM HEPES, pH 7.5, 100 mM NaCl, 2 mM MgCl2, 0.1% BSA, 2 mM TCEP, 0.05% Tween-20 (dilution buffer). 9.8 μL of this protein mixture was then mixed with the compounds in the assay plate and incubated for one hour at room temperature. Reaction mixtures with no compounds added were included as negative controls, while a His-tagged and biotinylated GST protein was included to track general assay performance. Nickel Chelate AlphaLISA Acceptor beads (cat. #AL108M, Perkin Elmer, MA) and AlphaScreen Streptavidin Donor beads (cat. #6760002, Perkin Elmer, Waltham, MA) were prepared separately in the dilution buffer as 4× stocks (80 ug/ml). The beads were then added to the assay plate sequentially with incubation at room temperature for 1-2 hours after each addition. Signals were recorded using EnVision 2104 Multilabel Plate Reader (Perkin Elmer, Waltham, MA) and titration curves were generated in GraphPad Prism (7.00, GraphPad, La Jolla, CA). Results are summarized in Table 2.

### Example 8: Measurement of Binary Interaction Between Compounds and DCAF15 Using Homogeneous Time Resolved Fluorescence (HTRF)

DCAF15 interactions with test compounds were monitored using HTRF competition assays (Cisbio, Bedford, MA). Test compounds were first collected in a 384-well microplate (cat. #781280, Grenier Bio-One, Monroe, NC) as 10 mM stocks in 100% dimethyl sulfoxide (DMSO). A second stock plate (cat. #781280, Grenier Bio-One, Monroe, NC) was prepared through 3-fold serial dilution in DMSO of the afore-mentioned stocks using a Mosquito HTS dispenser (mosquito@ HTS, SPT Labtech, Boston, MA). 0.2 μL of these diluted stocks were then dispensed into a 384-well Optiplate (cat. #6007290, Perkin Elmer, Waltham, MA) in duplicate wells, to assemble the assay plate. The tracer molecule containing an Alexa Fluor 647 probe was prepared in 25 mM HEPES pH 7.5, 100 mM NaCl, 0.1 mg/ml BSA, 0.005% Tween 20, 0.5 mM TCEP (dilution buffer) to 612 nM (2.04×). Separately, His-tagged DCAF15 complex was prepared at 32 nM (4×) in the dilution buffer, and mAb Anti-6His-Eu cryptate Gold (cat. #61H12KLA, Cisbio, Bedford, MA) at 4× dilution in the detection buffer (cat. #61DB9RDF, Cisbio, Bedford, MA). These two solutions were mixed and incubated for 15 min at room temperature. 9.8 μL of the tracer solution and 10 μL of the DCAF15/mAb Anti-6His-Eu cryptate Gold mixture were added sequentially to each well in the assay plate. Reaction mixtures with no DCAF15 added were included as positive controls. The final mixture was incubated for an hour at room temperature and spun down briefly before data collection at 615 nm and 666 nm using EnVision 2104 Multilabel Plate Reader (Perkin Elmer, Bedford, MA). Binding was estimated based on ratiometric analysis and titration curves were generated in GraphPad Prism (7.00, GraphPad, La Jolla, CA). Results are summarized in Table 2.

### Example 9: Western Blot Assay Procedure for Assessing Degradation of RBM39 in HCT116 Cells

HCT116 cells were used with passage numbers ranging from 13 to 25. Cells were seeded in 12-well plated at a density of 1 million cells per well in 1 mL cell culture medium containing McCoy's 5A medium (Cat. #16600082, ThermoFisher, Waltham, MA), 10% Fetal Bovine Serum (Cat. #26140, ThermoFisher, Waltham, MA) and 1% Pen-Strep (Cat. #. 15070063, ThermoFisher, Waltham, MA) and incubated overnight. The next morning, compounds were serially diluted (1:3, 9 points) in the medium separately and were prepared 2× the final assay concentration. Subsequently, 1.0 mL medium containing DMSO or the appropriate concentration of each compound was added to respective wells. The final DMSO concentration in each well was 0.1%. After 24 hour incubation with compounds, cell were harvested (approximately 90-100% confluency at the time of harvest). Cells were then washed once with 1 mL of ice cold Dulbecco's phosphate-buffered saline (Cat. #28374, ThermoFisher, Waltham, MA) and then lysed with 100 μl of lysis buffer containing 1×RIPA (Cat. #. 89900, ThermoFisher, Waltham, MA) and HALT protease and phosphatase inhibitor (Cat. #78442, ThermoFisher, Waltham, MA) on ice for 10 minutes with intermittent rocking. Lysates were then sonicated in a sonication bath (3 pulses of 10 sec each with 1 min incubation on ice between each pulse). Samples were then centrifuged for 20 min at 14,000 rpm for 20 min. Supernatants were then transferred to fresh microfuge tubes and stored frozen at −80° C. until use. Total protein quantitation in lysate samples was done using the BCA protein quantitation kit following the manufacturer's instructions. For polyacrylamide gel electrophoresis, each sample (15-30 μL total protein), was diluted with Laemmli sample buffer (Cat. #. 1610747, BioRad, Hercules, CA) by adding 9 μL of lysate to 3 μL of sample buffer containing 10% β-mercaptoethanol. Samples were then denatured at 95° C. for 5 minutes in a heat block, centrifuged at 14,000 rpm for 5 min, and placed on ice. Subsequently, 12 μL of each sample was loaded on 4-20% Tris/Glycine gels and electrophoresed in 1×Tris/Glycine SDS buffer at 80 (constant) volts for 2 h. Resolved samples were then transferred from gels to PVDF membrane using the BioRad Turbo-transfer unit with the Turbo default program (Cat. #. 1704150EDU, BioRad, Hercules, CA). For immunoblotting, membranes were first blocked for 1 hour at room temperature using Odyssey Blocking Buffer (Cat. #: 927-60001, Li-cor, Lincoln, NE) with continuous rocking. Membrane was then incubated with primary antibodies, anti-RBM39 (Sigma Cat. #: HPA01591, St. Louis, MO) and anti-R3-actin (Cell Signaling Cat. #3700S, Danvers, MA), diluted 1000 fold each, at 4° C. for overnight. Subsequently, membrane was washed three times for at room temperature with continuous rocking (5 minutes each wash) using TBS buffer containing 0.1% Tween-20. After the final wash, secondary antibodies, IRDye 680 RD Goat anti Mouse IgG H+L (Cat. #: 926-68070, Li-cor, Lincoln, NE), and IRDye 800CW Goat anti Rabbit IgG H+L (Cat. #: 926-32211, Li-cor, Lincoln, NE), each diluted 10,000-fold, were added to the membranes and incubated at room temperature for 1 h with continuous rocking. Membranes were then washed 3 times with lxTBS and scanned using Odessey CLx imaging system (Li-cor, Lincoln, NE) using default settings. Band intensity in images was quantified using the Image Studio Lite software (Version 5.2). Signal intensity for RBM-39 specific bands were normalized using the signal intensity for P3-actin-specific band, and percent degradation was assessed by comparing RBM39 signal in compound-treated vs. DMSO-treated samples. Results are summarized in Table 2.

TABLE 2
Biological Data

| | | | RBM39 |
|---|---|---|---|
| | AlphaLISA | HTRF | Degradation: |
| | ternary - | binary - | IC₅₀ (uM) |
| | DCAF15/RBM39: | DCAF15: | [Incubation |
| Compound | Mean EC₅₀ (M)* | Mean IC₅₀* | Time: 4 hours]* |
| Compound 2 | ++ | | |
| Compound 5 | ++ | | |
| Compound 7 | +++ | ++ | |
| Compound 8 | +++ | ++ | |
| Compound 9 | ++ | − | |
| Compound 10 | +++ | ++ | |
| Compound 11 | +++ | ++ | |
| Compound 12 | ++ | ++ | |
| Compound 14 | ++ | ++ | |
| Compound 15 | ++ | ++ | |
| Compound 16 | ++ | ++ | |
| Compound 17 | +++ | ++ | +++ |
| Compound 18 | +++ | ++ | |
| Compound 19 | ++ | + | |
| Compound 21 | ++ | | |
| Compound 22 | +++ | ++ | |
| Compound 23 | ++ | ++ | |
| Compound 24 | ++ | − | |
| Compound 25 | +++ | ++ | |
| Compound 28 | + | | |
| Compound 29 | ++ | + | |
| Compound 30 | ++ | | |
| Compound 31 | +++ | ++ | |
| Compound 33 | +++ | | |
| Compound 34 | ++ | ++ | |
| Compound 35 | ++ | ++ | |
| Compound 36 | +++ | ++ | |
| Compound 37 | +++ | ++ | |
| Compound 38 | ++ | ++ | |
| Compound 39 | ++ | | |
| Compound 40 | +++ | | |
| Compound 41 | +++ | ++ | |
| Compound 42 | +++ | ++ | +++ |
| Compound 43 | +++ | | |
| Compound 44 | +++ | | +++ |
| Compound 45 | +++ | + | |
| Compound 46 | ++ | | |
| Compound 47 | +++ | ++ | |
| Compound 48 | +++ | ++ | |
| Compound 49 | +++ | ++ | |

*+++ ≤1 μM; <1 μM ++ ≤10 μM; <10 μM + ≤100 μM; −>100 μM

1. A compound of Formula I 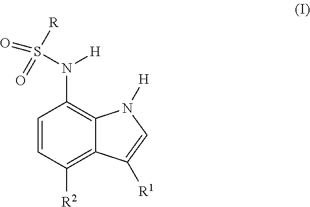
or a pharmaceutically acceptable salt thereof, wherein:
R is an optionally substituted -5,6; -6,5; -6,6; or -6,7 bicyclic ring system;
R¹ is selected from the group consisting of chloro, bromo, fluoro, and iodo; and
R² is selected from the group consisting of H, chloro, fluoro, and methyl.

2. The compound of claim 1, wherein
R is 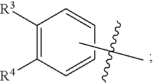
and
R³ and R⁴, together with the carbon atoms to which they are attached, form a 5 to 7-membered optionally substituted carbocyclylic, heterocyclyic, heteroaryl, or aryl ring.

3. The compound of claim 1, or a pharmaceutically acceptable salt thereof, wherein
R is 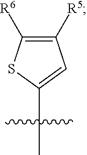
and
R⁵ and R⁶, together with the carbon atoms to which they are attached, form an optionally substituted 6-membered heterocyclylic or aryl ring.

4. The compound of claim 1, or a pharmaceutically acceptable salt thereof, wherein R is selected from the group consisting of optionally substituted 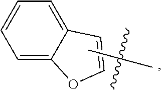
optionally substituted 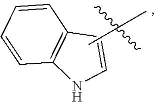
optionally substituted 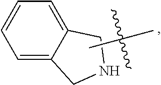
optionally substituted 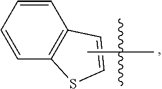
optionally substituted 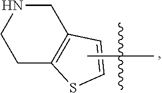
optionally substituted, 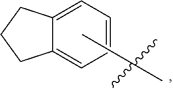
optionally substituted, 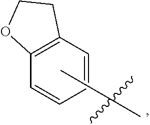
optionally substituted 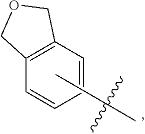
optionally substituted 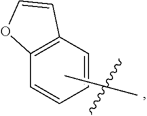
optionally substituted 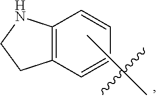
optionally substituted 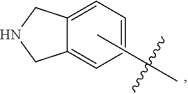
optionally substituted 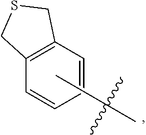
optionally substituted 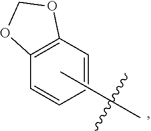
optionally substituted 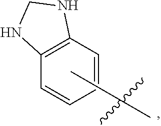
optionally substituted 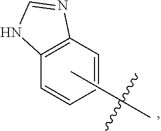
optionally substituted 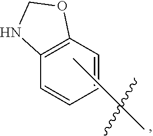
optionally substituted 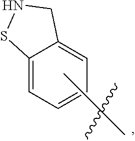
optionally substituted 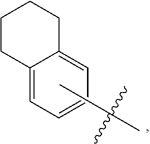
optionally substituted 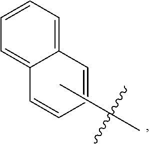
optionally substituted 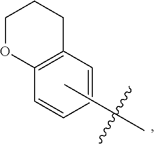
optionally substituted 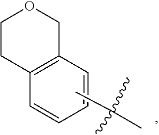
optionally substituted 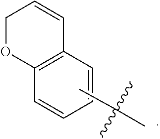
optionally substituted 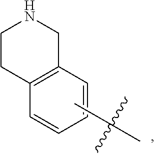
optionally substituted 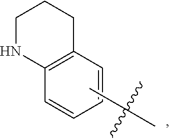
optionally substituted 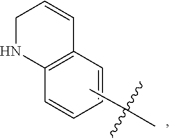
optionally substituted 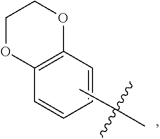
optionally substituted 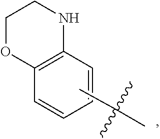
optionally substituted 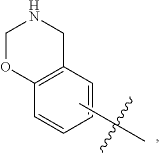
optionally substituted 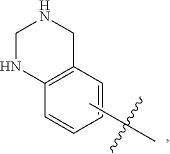
optionally substituted 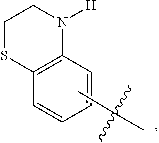
optionally substituted 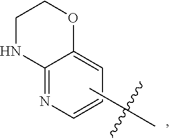
optionally substituted 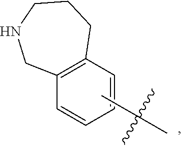
optionally substituted 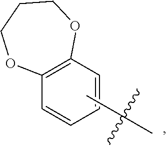
optionally substituted 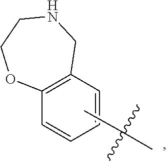
optionally substituted

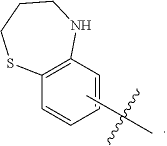

5. The compound according to claim 1 or 4, or a pharmaceutically acceptable salt thereof, wherein R is selected from the group consisting of

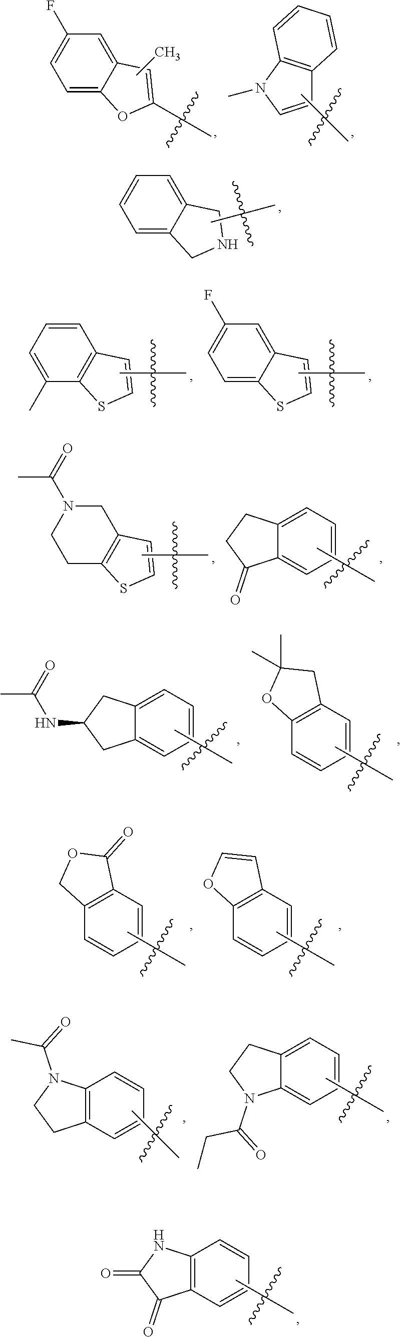

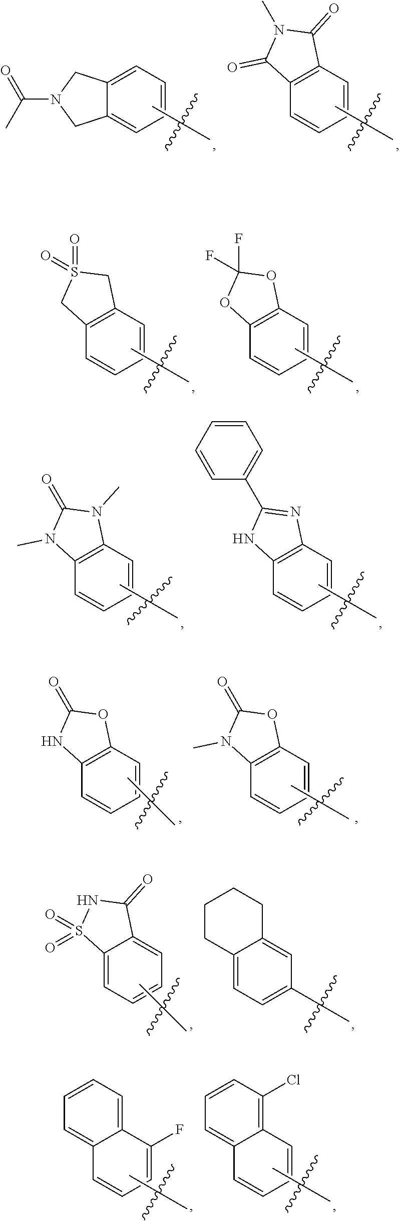

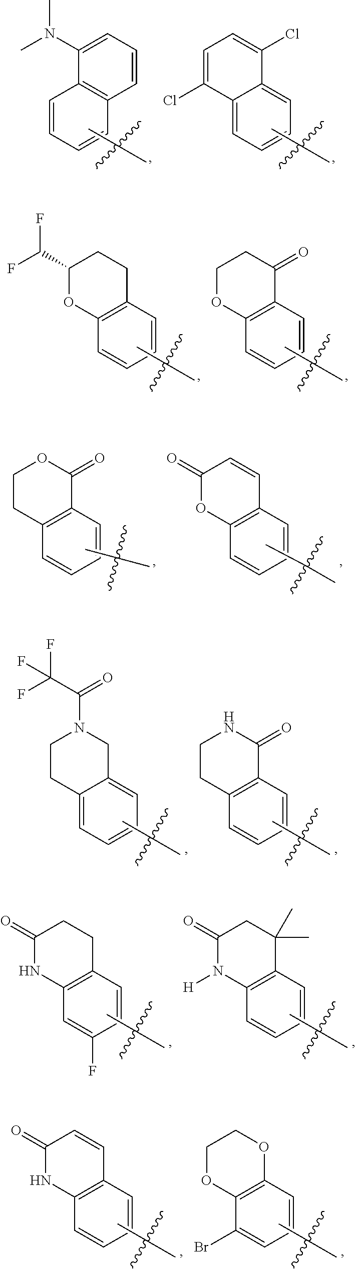

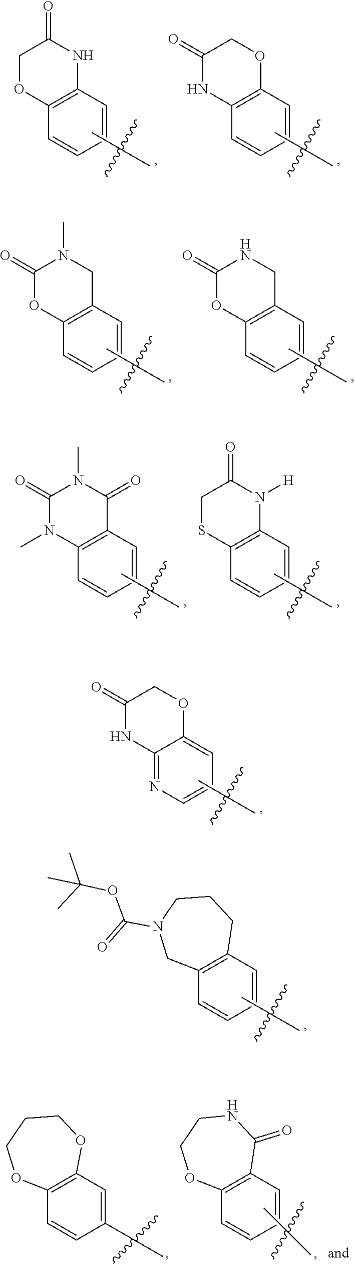

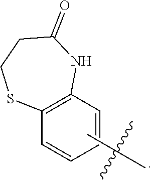

6. The compound according to an one of claims 1 to 5, or a pharmaceutically acceptable salt thereof, wherein each of R¹ and R² is Cl.

7. The compound according to claim 1, or a pharmaceutically acceptable salt thereof, wherein the compound is selected from the group consisting of 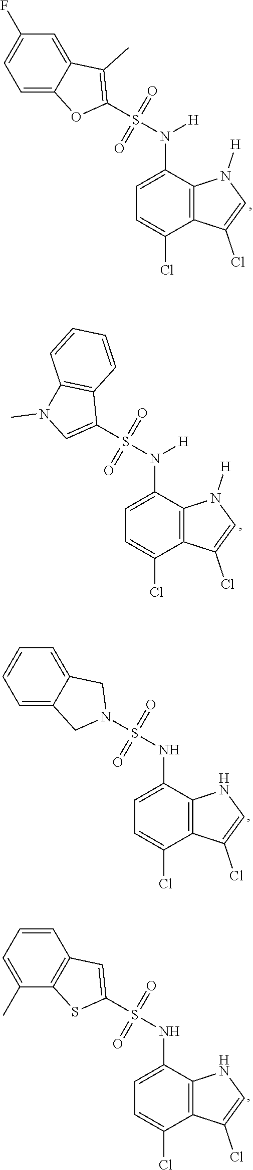 [SMILES: Cc1c(S(=O)(=O)Nc2ccc(Cl)c3c(Cl)c[nH]c23)oc2ccc(F)cc12 ; Cn1cc(S(=O)(=O)Nc2ccc(Cl)c3c(Cl)c[nH]c23)c2ccccc21 ; O=S(=O)(Nc1ccc(Cl)c2c(Cl)c[nH]c12)N1Cc2ccccc2C1 ; Cc1cccc2cc(S(=O)(=O)Nc3ccc(Cl)c4c(Cl)c[nH]c34)sc12] 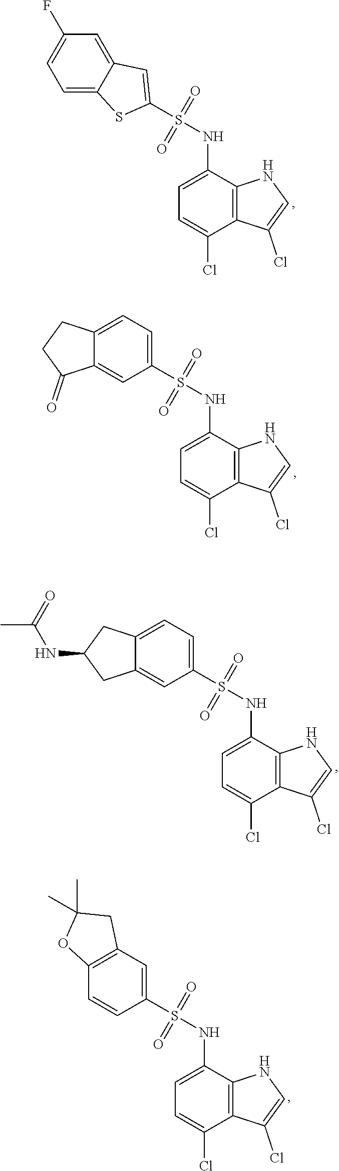 [SMILES: O=S(=O)(Nc1ccc(Cl)c2c(Cl)c[nH]c12)c1cc2cc(F)ccc2s1 ; O=C1CCc2ccc(S(=O)(=O)Nc3ccc(Cl)c4c(Cl)c[nH]c34)cc21 ; CC(=O)N[C@H]1Cc2ccc(S(=O)(=O)Nc3ccc(Cl)c4c(Cl)c[nH]c34)cc2C1 ; CC1(C)Cc2cc(S(=O)(=O)Nc3ccc(Cl)c4c(Cl)c[nH]c34)ccc2O1] 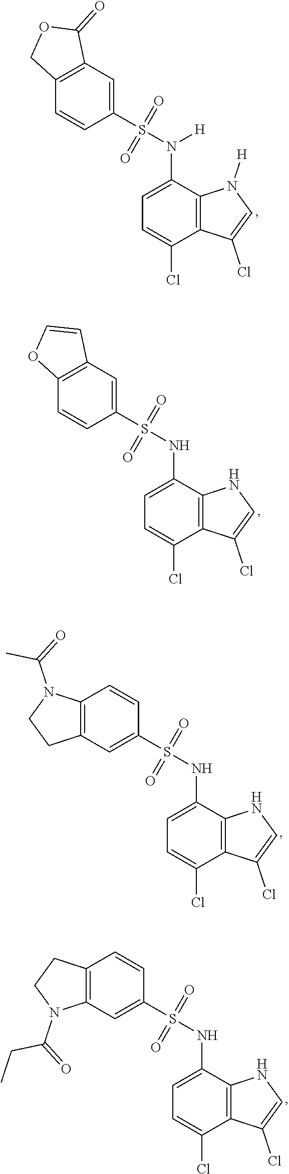 [SMILES: O=C1OCc2ccc(S(=O)(=O)Nc3ccc(Cl)c4c(Cl)c[nH]c34)cc21 ; O=S(=O)(Nc1ccc(Cl)c2c(Cl)c[nH]c12)c1ccc2occc2c1 ; CC(=O)N1CCc2cc(S(=O)(=O)Nc3ccc(Cl)c4c(Cl)c[nH]c34)ccc21 ; CCC(=O)N1CCc2ccc(S(=O)(=O)Nc3ccc(Cl)c4c(Cl)c[nH]c34)cc21] 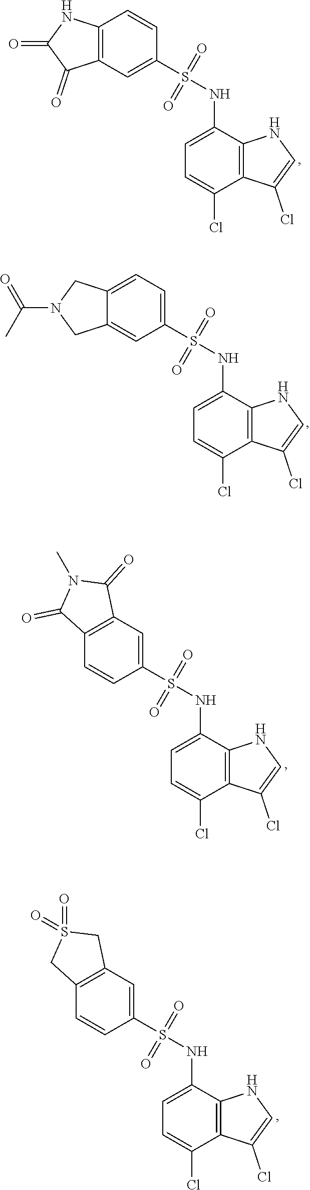 [SMILES: O=C1Nc2ccc(S(=O)(=O)Nc3ccc(Cl)c4c(Cl)c[nH]c34)cc2C1=O ; CC(=O)N1Cc2ccc(S(=O)(=O)Nc3ccc(Cl)c4c(Cl)c[nH]c34)cc2C1 ; CN1C(=O)c2ccc(S(=O)(=O)Nc3ccc(Cl)c4c(Cl)c[nH]c34)cc2C1=O ; O=S1(=O)Cc2ccc(S(=O)(=O)Nc3ccc(Cl)c4c(Cl)c[nH]c34)cc2C1] 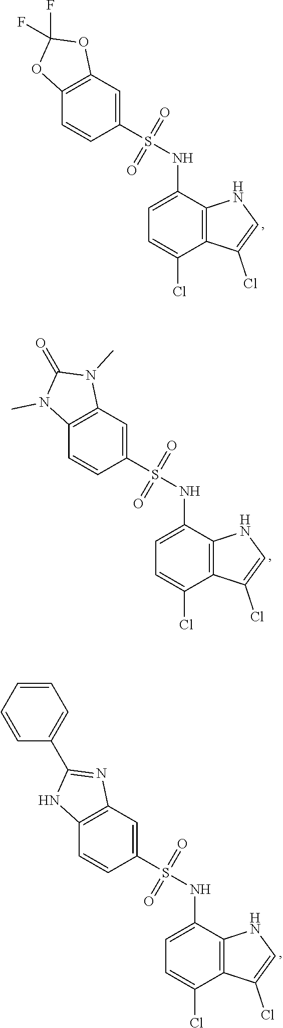 [SMILES: O=S(=O)(Nc1ccc(Cl)c2c(Cl)c[nH]c12)c1ccc2c(c1)OC(F)(F)O2 ; Cn1c(=O)n(C)c2cc(S(=O)(=O)Nc3ccc(Cl)c4c(Cl)c[nH]c34)ccc21 ; O=S(=O)(Nc1ccc(Cl)c2c(Cl)c[nH]c12)c1ccc2[nH]c(-c3ccccc3)nc2c1] 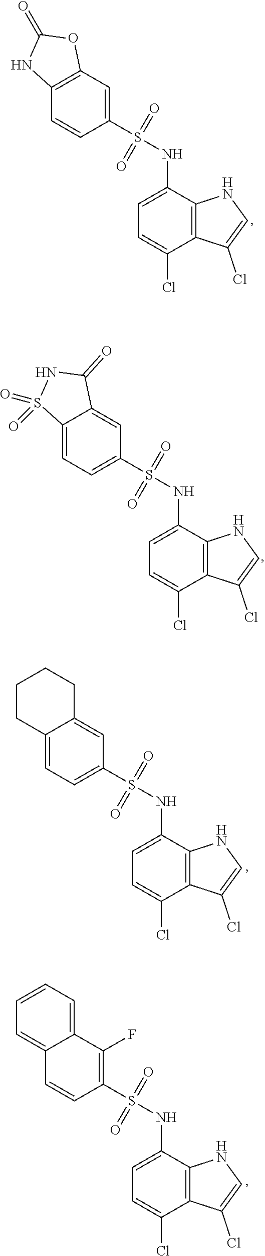 [SMILES: O=c1[nH]c2ccc(S(=O)(=O)Nc3ccc(Cl)c4c(Cl)c[nH]c34)cc2o1 ; O=C1NS(=O)(=O)c2ccc(S(=O)(=O)Nc3ccc(Cl)c4c(Cl)c[nH]c34)cc21 ; O=S(=O)(Nc1ccc(Cl)c2c(Cl)c[nH]c12)c1ccc2c(c1)CCCC2 ; O=S(=O)(Nc1ccc(Cl)c2c(Cl)c[nH]c12)c1ccc2ccccc2c1F] 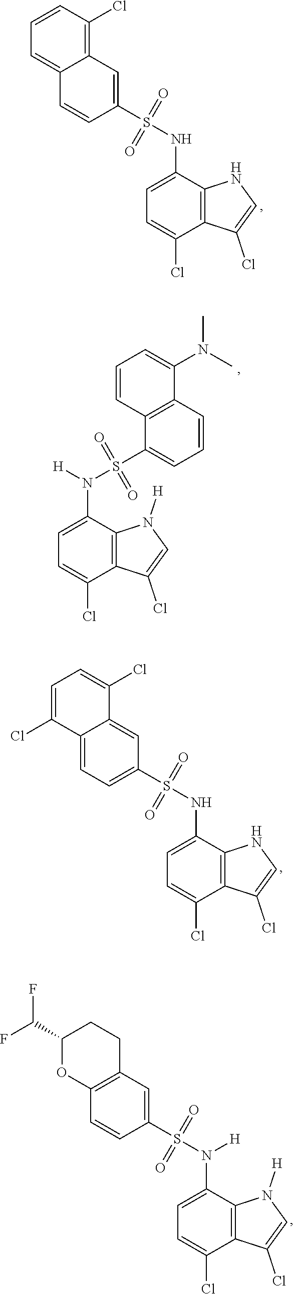 [SMILES: O=S(=O)(Nc1ccc(Cl)c2c(Cl)c[nH]c12)c1ccc2cccc(Cl)c2c1 ; CN(C)c1cccc2c(S(=O)(=O)Nc3ccc(Cl)c4c(Cl)c[nH]c34)cccc12 ; O=S(=O)(Nc1ccc(Cl)c2c(Cl)c[nH]c12)c1ccc2c(Cl)ccc(Cl)c2c1 ; O=S(=O)(Nc1ccc(Cl)c2c(Cl)c[nH]c12)c1ccc2c(c1)CC[C@@H](C(F)F)O2] 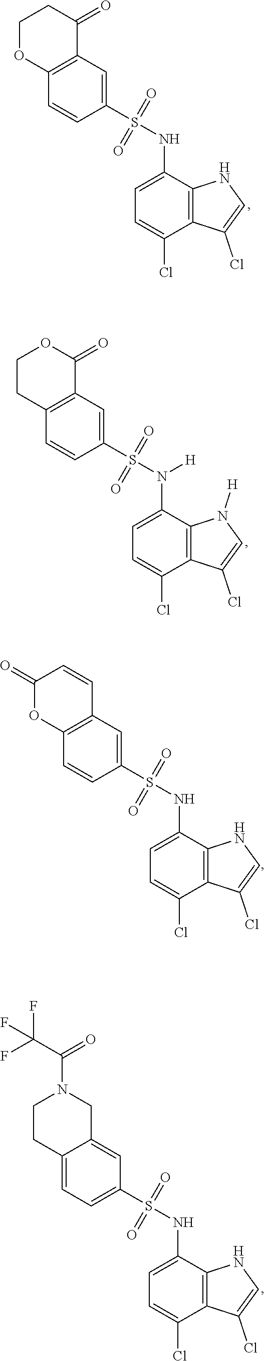 [SMILES: O=C1CCOc2ccc(S(=O)(=O)Nc3ccc(Cl)c4c(Cl)c[nH]c34)cc21 ; O=C1OCCc2ccc(S(=O)(=O)Nc3ccc(Cl)c4c(Cl)c[nH]c34)cc21 ; O=c1ccc2cc(S(=O)(=O)Nc3ccc(Cl)c4c(Cl)c[nH]c34)ccc2o1 ; O=C(N1CCc2ccc(S(=O)(=O)Nc3ccc(Cl)c4c(Cl)c[nH]c34)cc2C1)C(F)(F)F] 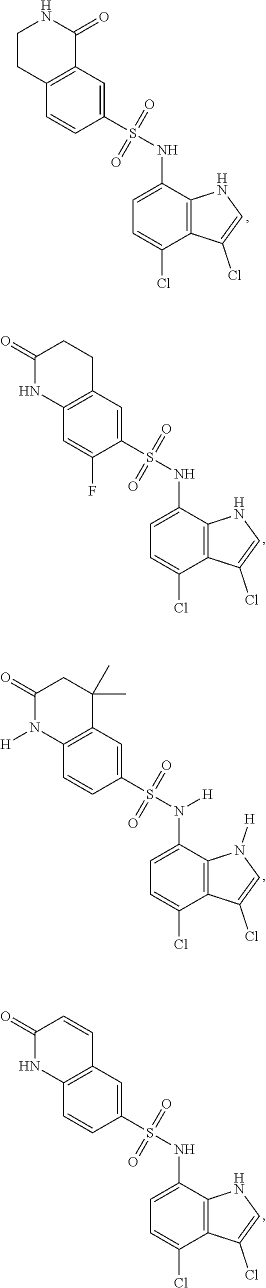 [SMILES: O=C1NCCc2ccc(S(=O)(=O)Nc3ccc(Cl)c4c(Cl)c[nH]c34)cc21 ; O=C1CCc2cc(S(=O)(=O)Nc3ccc(Cl)c4c(Cl)c[nH]c34)c(F)cc2N1 ; CC1(C)CC(=O)Nc2ccc(S(=O)(=O)Nc3ccc(Cl)c4c(Cl)c[nH]c34)cc21 ; O=c1ccc2cc(S(=O)(=O)Nc3ccc(Cl)c4c(Cl)c[nH]c34)ccc2[nH]1] 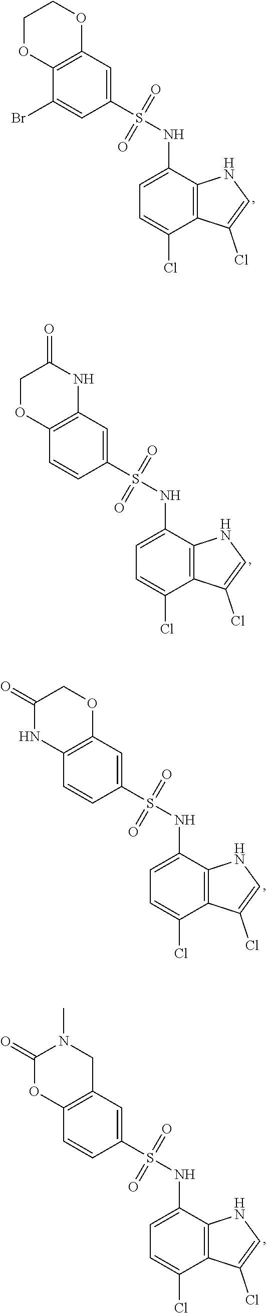 [SMILES: O=S(=O)(Nc1ccc(Cl)c2c(Cl)c[nH]c12)c1cc(Br)c2c(c1)OCCO2 ; O=C1COc2ccc(S(=O)(=O)Nc3ccc(Cl)c4c(Cl)c[nH]c34)cc2N1 ; O=C1COc2cc(S(=O)(=O)Nc3ccc(Cl)c4c(Cl)c[nH]c34)ccc2N1 ; CN1Cc2cc(S(=O)(=O)Nc3ccc(Cl)c4c(Cl)c[nH]c34)ccc2OC1=O] 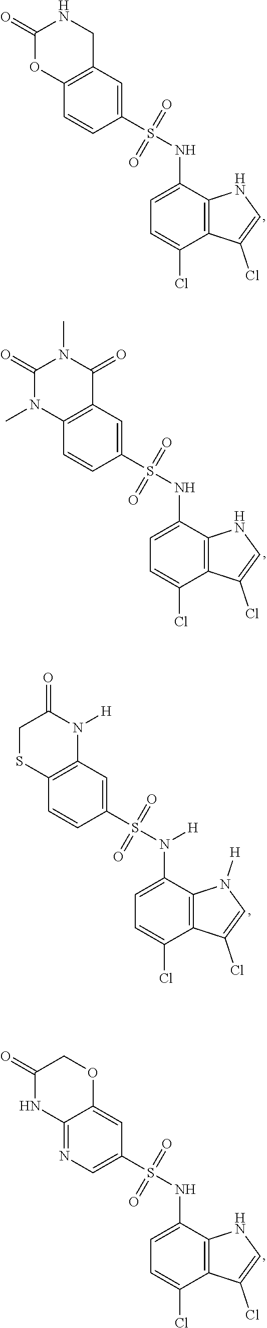 [SMILES: O=C1NCc2cc(S(=O)(=O)Nc3ccc(Cl)c4c(Cl)c[nH]c34)ccc2O1 ; Cn1c(=O)c2cc(S(=O)(=O)Nc3ccc(Cl)c4c(Cl)c[nH]c34)ccc2n(C)c1=O ; O=C1CSc2ccc(S(=O)(=O)Nc3ccc(Cl)c4c(Cl)c[nH]c34)cc2N1 ; O=C1COc2cc(S(=O)(=O)Nc3ccc(Cl)c4c(Cl)c[nH]c34)cnc2N1] 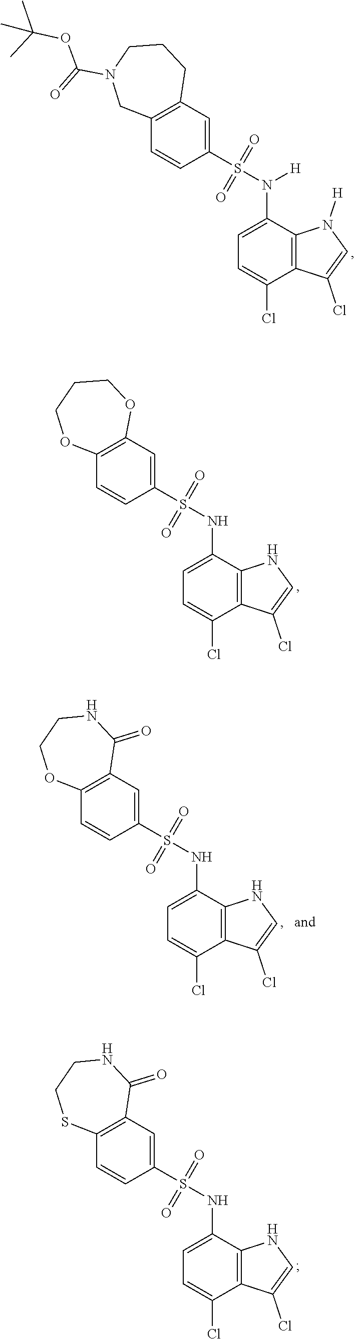 [SMILES: CC(C)(C)OC(=O)N1CCCc2cc(S(=O)(=O)Nc3ccc(Cl)c4c(Cl)c[nH]c34)ccc2C1 ; O=S(=O)(Nc1ccc(Cl)c2c(Cl)c[nH]c12)c1ccc2c(c1)OCCCO2 ; O=C1NCCOc2ccc(S(=O)(=O)Nc3ccc(Cl)c4c(Cl)c[nH]c34)cc21 ; O=C1NCCSc2ccc(S(=O)(=O)Nc3ccc(Cl)c4c(Cl)c[nH]c34)cc21]
or a pharmaceutically acceptable salt thereof.

8. A pharmaceutical composition comprising a therapeutically effective amount of a compound of any one of claims 1 to 7, or a pharmaceutically acceptable salt thereof; and a pharmaceutically acceptable carrier.

9. A method of inducing degradation of a protein, comprising contacting the protein with an effective amount of a compound according to any one of claims 1-7, or a pharmaceutically acceptable salt thereof, or the composition according to claim 8.

10. The method of claim 9, wherein the compound promotes the formation of a complex between the protein and a substrate recognition subunit of an ubiquitin E3 ligase.

11. The method of claim 9, wherein the compound binds to the ubiquitin E3 ligase.

12. The method of claim 9, wherein the substrate recognition subunit is DCAF15.

13. The method of claim 9, wherein the protein is RBM-39.

14. The method of claim 9, wherein the substrate recognition subunit is DCAF15 and the protein is RBM-39.

15. A method of inducing degradation of a protein, in a patient in need thereof, comprising administering to the patient, a therapeutically effective amount of a compound according to any one of claims 1 to 7, or a pharmaceutically acceptable salt thereof, or the composition according to claim 8.

16. A method of treating a disease or disorder that results from abnormal activity of a target protein in a subject, comprising administering to the subject, a therapeutically effective amount of a compound according to any one of claims 1 to 7, or a pharmaceutically acceptable salt thereof, or the composition according to claim 8, wherein the compound or salt induces degradation of the protein thereby treating the disease or disorder.

17. The method of claim 16, wherein the disease or disorder results directly from abnormal activity of a target protein.

18. The method of claim 16, wherein the disease or disorder results indirectly from the abnormal activity of the target protein.

19. A method of treating a disease or disorder that results from abnormal expression of a gene that encodes a target protein in a subject, comprising administering to the subject, a therapeutically effective amount of a compound according to any one of claims 1 to 7, or a pharmaceutically acceptable salt thereof, or the composition according to claim 8, wherein the compound or salt induces degradation of the protein thereby treating the disease or disorder.
